(19) United States
(12) Patent Application Publication
(10) Pub. No.: US 2005/0143388 A1
(43) Pub. Date: Jun. 30, 2005

(54) Nitric oxide donors for treatment of disease and injury

(76) Inventor: Michael Chopp, Detroit, MI (US)

Correspondence Address: Amy E Rinaldo;Kohn & Associates, Suite 410, 30500 Northwestern Highway, Farmington Hills, MI, 48334, US

(21) Appl. No.: 10/500,694

(22) PCT Filed: Jan. 6, 2003
(86) PCT No.: PCT/US03/00323

Related U.S. Application Data
(60) Provisional application No. 60/345,478, filed on Jan. 4, 2002.

(51) Int. Cl. A61K 31/519

(52) U.S. Cl. 514/252.16; 514/262.1

(57) ABSTRACT

A method of promoting neurogenesis by administering a therapeutic amount of a phosphodiesterase inhibitor compound is a patient in need of neurogenesis promotion. A compound for providing neurogenesis having an effective amount of a phosphodiesterase inhibitor sufficient to promote neurogenesis. A phosphodiesterase inhibitor for promoting neurogenesis. A method of augmenting the production of brain cells and facilitating cellular structual and receptor changes by administering an effective amount of a phosphodiesterase inhibitor compound to a site in need of augmentation. A method of increasing both neurological and cognitive function by administering an effective amount of a phosphodiesterase inhibitor compound to a patient.

### BACKGROUND OF THE INVENTION

1. Technical Field

The present invention relates to treatments of disease and injury. More specifically, the present invention relates to methods and compounds including nitric oxide donors and cell therapy for the treatment of disease and injury.

2. Background Art

Stroke is the third most common cause of death in the adult population of the United States, and is a major cause of disability. Stroke occurs when a section of the brain becomes infarcted, resulting in death of brain tissue from interruption of cerebral blood supply. Cerebral infarcts associated with acute stroke cause sudden and dramatic neurological impairment. Other neurological diseases also result in the death of tissue and neurological impairment.

Pharmacological interventions have attempted to maximize the blood flow to stroke affected brain areas that might be able to survive, but clinical effectiveness has proven elusive. As stated in Harrison's Principles of Internal Medicine (9^{th} Ed., 1980, p. 1926), “despite experimental evidence that . . . [cerebral vasodilators] increase the cerebral blood flow, as measured by the nitrous oxide method, they have not proved beneficial in careful studies in human stroke cases at the stage of transient ischemic attacks, thrombosis-in-evolution, or in the established stroke. This is true of nicotinic acid, Priscoline, alcohol, papaverine, and inhalation of 5% carbon dioxide. . . . In opposition to the use of these methods is the suggestion that vasodilators are harmful rather than beneficial, since by lowering the systemic blood pressure they reduce the intracranial anastomotic flow, or by dilating blood vessels in the normal parts of the brain they steal blood from the infarct.”

Additionally, diseases of the cardiovascular system are a leading worldwide cause of mortality and morbidity. For example, heart failure has been increasing in prevalence. Heart failure is characterized by an inability of the heart to deliver sufficient blood to the various organs of the body. Current estimates indicate that over 5 million Americans carry the diagnosis of heart failure with nearly 500,000 new cases diagnosed each year and 250,000 deaths per year attributed to this disease. Despite significant therapeutic accomplishments in the past two decades, heart failure continues to increase in incidence reaching epidemic proportions and representing a major economic burden in developed countries.

Heart failure is a clinical syndrome characterized by distinctive symptoms and signs resulting from disturbances in cardiac output or from increased venous pressure. Moreover, heart failure is a progressive disorder whereby the function of the heart continues to deteriorate over time despite the absence of adverse events. Thus, due to heart failure, inadequate cardiac output results.

Generally, there are two types of heart failure. Right heart failure is the inability of the right side of the heart to pump venous blood into pulmonary circulation. A back-up of fluid in the body occurs and results in swelling and edema. Left heart failure is the inability of the left side of the heart to pump blood into systemic circulation. Back-up behind the left-ventricle then causes accumulation of fluid in the lungs.

The main resulting effect of heart failure is fluid congestion. If the heart becomes less efficient as a pump, the body attempts to compensate for it by using hormones and neural signals, for example, to increase blood volume.

Heart failure has numerous causes. For example, disease of heart tissue results in dead myocardial cells that no longer function. Progression in left ventricular dysfunction has been attributed, in part, to ongoing loss of these cardiomyocytes.

There have been numerous methods of treating and preventing heart failure. For example, stem cells have been used to regenerate cardiac cells in acute cardiac ischemia and/or infarction or injury in animal models. In one particular example, viable marrow stromal cells isolated from donor leg bones were culture-expanded, labeled, and then injected into the myocardium of isogenic adult rat recipients. After harvesting the hearts from 4 days to 12 weeks after implantation, the implantation sites were examined and it was found that implanted stromal cells show the growth potential in a myocardial environment. (Wang, et. al.)

Cardiomyocytes have been shown to differentiate in vitro from pluripotent embryonic stem (ES) cells of line D3 via embryo-like aggregates (embryoid bodies). The cells were characterized by the whole-cell patch-clamp technique, morphology, and gene expression analogy during the entire differentiation period. (Maltsev, et. al., 1994) Additionally, pluripotent mouse ES cells were capable to differentiate into cardiomyocytes expressing major features of mammalian heart (Maltsev, et. al., 1993).

Stem cells, regardless of their origin (embryonic, bone marrow, skeletal muscle, etc.), have the potential to differentiate into various, if not all, cell types of the body. Stem cells are able to differentiate into functional cardiac myocytes. Thus, the development of stem cell-based therapies for treating heart failure has many advantages over existing conventional therapies.

Accordingly, there is a need for a method of treating patients having disease or injury by combining cell therapy and the use of a nitric oxide donor.

### SUMMARY OF THE INVENTION

According to the present invention, there is provided a method of promoting neurogenesis by administering a therapeutic amount of a phosphodiesterase inhibitor compound to a patient in need of neurogenesis promotion. Also provided is a compound for providing neurogenesis having an effective amount of a phosphodiesterase inhibitor sufficient to promote neurogenesis. A phosphodiesterase inhibitor for promoting neurogenesis is also provided. Further, a method of augmenting the production of brain cells and facilitating cellular structural and receptor changes by administering an effective amount of a phosphodiesterase inhibitor compound to a site in need of augmentation is provided. There is provided a method of increasing both neurological and cognitive function by administering an effective amount of a phosphodiesterase inhibitor compound to a patient.

### BRIEF DESCRIPTION OF THE DRAWINGS

Other advantages of the present invention are readily appreciated as the same becomes better understood by reference to the following detailed description when considered in connection with the accompanying drawings wherein:

FIGS. 1A-D show cerebral vascular perimeters;

FIGS. 2A-C show proliferated cerebral endothelial cells;

FIGS. 3A-C show DETANONOate induces angiogenesis, as analyzed with three-dimensional images;

FIGS. 4A-E show DETANONOate induces in vitro angiogenesis;

FIG. 5 shows a bar graph that shows quantitative data of Sildenafil-induced capillary-like tube formation;FIGS. 6A-I are photographs showing the effects of treating cells with sildenafil;

FIGS. 7A and B are graphs that show levels of cGMP in the cerebellum and cortex respectively after treatment with sildenafil versus controls in nonischemic rats;

FIG. 8 is a graph that shows localized CBF in rats treated with sildenafil versus controls;

FIGS. 9A and B are graphs that show the results of the adhesive-removal test and mNSS test respectively;

FIGS. 10A and B are a photograph and graph respectively that shows the results of treatment of BrdU positive cells in the SVZ using the therapy of the present invention;

FIGS. 11A and B are a photograph and graph respectively that shows the results of treatment of BrdU positive cells in the vessels using the therapy of the present invention;

FIGS. 12A-C are photographs that show that the treatment of the present invention induces endothelial tube formation by brain-derived endothelial cells compared with controls;

FIG. 13 is a graph that shows that the treatment of the present invention increased VEGF secretion compared to controls;

FIGS. 14A-G are photographs shows the results of the therapy of the present invention;

FIGS. 15A-C are bar graphs that show the number of BrdU immunoreactive cells in the dentate gyrus (FIG. 15A), in the SVZ (FIG. 15B), and in the OB (FIG. 15C) in non-ischemic young adult rats at 14 ( [symbol] ) and 42 ( [symbol] ) days after treatment with DETA/NONOate or saline;

FIGS. 16A-C are bar graphs that show the number of BrdU immunoreactive cells in the dentate gyrus (FIG. 16A), in the SVZ (FIG. 16B), and in the OB (FIG. 16C) in non-ischemic aged rats at 14 ( [symbol] ) and 42 ( [symbol] ) days after treatment with DETA/NONOate or saline;

FIGS. 17A-D show the effect of SNAP treatment on infarct volume (FIG. 17A), rotarod (FIG. 17B) and adhesive removal (FIG. 17C) tests as well as animal body weight (FIG. 17D); and

FIGS. 18A andB are photographs that show RT-PCR of PDE5A1 (FIG. 18A) and PDE5A2 (FIG. 18B) mRNA in the cortex of non-ischemic rats (N in FIG. 18A and FIG. 18B) and the ipsilateral cortex of rats 2 hours to 7 days after ischemia.

### DESCRIPTION OF THE INVENTION

Generally, the present invention provides a method and compound for treating injury or disease in multi-organ systems with a combination of cellular therapy and a nitric oxide donor or PDE inhibitor. This combination therapy increases the effectiveness of both therapies without increasing any risk to a patient. The benefit of the therapy is that is augments organ plasticity by inducing neurogenesis, angiogenesis, and alterations in parenchymal cell structure and function. Additionally, because of the synergistic effect, lower doses of each therapy can be given, thereby limiting any side effects or harmful effects of the drugs which can otherwise manifest themselves. Alternatively, the PDE inhibitor alone can be administered for treatment.

By “PDE inhibitor” it is meant a compound that inhibits PDE. An example of such a compound is sildenafil (Viagra™). A PDE inhibitor is an agent that reduces (e.g. selectively reduces) or eliminates the activity of a phosphodiesterase, such as PDE1-10 (e.g. type V phosphodiesterase, type 10 phosphosdiesterase), and any other phosphodiesterases. In the context of the methods and compositions of the present invention, the phosphodiesterase inhibitors include salts, esters, amides, prodrugs and other derivatives of the active agents (e.g. the PDE). The phosphodiesterase inhibitor amplifies the effects of any NO produced. The Phosphodiesterase inhibitor can be used to produce vasodilation and improvement in vascular function.

Examples of these inhibitor compounds include, but are not limited to rolipram,, theophylline, pentoxifylline, cGMP, zaprinast, IBMX, milrinone, 5-(2-ethoxy-5-morpholinoacetylphenyl)-1-methyl-3-n-propyl-1,6-dihydro-7H-pyrazolo[4,3-d]pyrimidin-7-one, 5-(5-morpholinoacetyl-2-n-propoxyphenyl)-1-methyl-3-n-propyl-1,6-dihydro-7-H-pyrazolo[4,3-d]pyrimidin-7-one, 5-[2-ethoxy-5-(4-methyl-1-piperazinylsulfonyl)-phenyl]1-methyl-3-n-propyl-1,6-dihydro-7-H-pyrazolo[4,3-d]pyrimidin-7-one, 5-[2-allyloxy-5-(4-methyl-1-piperazinylsulfonyl)-phenyl)-1-methyl-3-n-propyl-1,6-dihydro-7H-pyrazolo[4,3-d]pyrimidin-7-one, 5-[2-ethoxy-5-[4-(2-propyl)-1-piperazinylsulfonyl)-phenyl]-1-methyl-3-n-propyl-1,6-dihydro-7-H-pyrazolo[4,3-d]pyrimidin-7-one, 5-[2-ethoxy-5-[4-(2-hydroxyethyl)-1-piperazinylsulfonyl)phenyl]-1-methyl-3-n-propyl-1,6-dihydro-7-H-pyrazolo[4,3-d]pyrimidin-7-one, 5-[5-[4-(2-hydroxyethyl)-1-piperazinylsulfonyl]-2-n-propoxyphenyl]-1-methyl-3-n-propyl-1,6-dihydro-7-H-pyrazolo[4,3-d]pyrimidin-7-one, 5-[2-ethoxy-5-(4-methyl-1-piperazinylcarbonyl)phenyl]-1-methyl-3-n-propyl-1,6-dihydro-7-H-pyrazolo[4,3-dlpyrimidin-7-one, and 5-[2-ethoxy-5-(1-methyl-2-imidazolyl)phenyl]-1-methyl-3-n-propyl-1,6-dihydro-7-H-pyrazolo[4,3-d]pyrimidin-7-one.

The phosphodiesterase inhibitors also can include griseolic acid derivatives, 2-phenylpurinone derivatives, phenylpyridone derivatives, fused and condensed pyrimidines, pyrimidopyrimidine derivatives, purine compounds, quinazoline compounds, phenylpyrimidinone derivative, imidazoquinoxalinone derivatives or aza analogues thereof, phenylpyridone derivatives, and others. Specific examples of the phosphodiesterase inhibitors include 1,3-dimethyl-5-benzylpyrazolo[4,3-d]pyrimidine-7-one, 2-(2-propoxyphenyl)-6-purinone, 6-(2-propoxyphenyl)-1,2-dihydro-2-oxypyridine-3-carboxamide, 2-(2-propoxyphenyl)-pyrido[2,3-d]pyrimid4(3H)-one, 7-methylthio-4-oxo-2-(2-propoxyphenyl)-3,4-dihydro-pyrimido[4,5-d]pyrimidine, 6-hydroxy-2-(2-propoxyphenyl)pyrimidine-4-carboxamide, 1-ethyl-3-methylimidazo[1,5a]quinoxalin-4(5H)-one, 4-phenylmethylamino-6-chloro-2-(1-imidazoloyl)quinazoline, 5-ethyl-8-[3-(N-cyclohexyl-N-methylcarbamoyl)-propyloxy]-4,5-dihydro-4-oxo-pyrido[3,2-e]-pyrrolo[1,2-a]pyrazine, 5′-methyl-3′-(phenylmethyl)-spiro[cyclopentane-1,7′(8′H)-(3′H)-imidazo[2,1b]purin]4′(5′H)-one, 1-[6-chloro-4-(3,4-methylenedioxybenzyl)-aminoquinazolin-2-yl)piperidine-4-carboxylic acid, (6R,9S)-2-(4-trifluoromethyl-phenyl)methyl-5-methyl-3,4,5,6a,7,8,9,9a-octa hydrocyclopent[4,5]-midazo[2,1-b]-purin-4-one, 1t-butyl-3-phenylmethyl-6-(4-pyridyl)pyrazolo[3,4-d]-pyrimid-4-one, 1-cyclopentyl-3-methyl-6-(4-pyridyl)-4,5-dihydro-1H-pyrazolo[3,4-d]pyrimid-4-one, 2-butyl-1-(2-chlorobenzyl)6-ethoxycarbonylbenzimidaole, and 2-(4-carboxypiperidino)-4-(3,4-methylenedioxybenzyl)amino-6-nitroquinazoline, and 2-phenyl-8-ethoxycycloheptimidazole.

Still other type V phosphodiesterase inhibitors useful in conjunction with the present invention include: IC-351 (ICOS); 4-bromo-5-(pyridylmethylamino)-6-[3-(4-chlorophenyl)propoxy]-3(2H)pyridazinone; 1-[4-[(1,3-benzodioxol-5-ylmethyl)amiono]-6-chloro-2-quinazolinyl]-4-piperidine-carboxylic acid, monosodium salt; (+)-cis-5,6a,7,9,9,9a-hexahydro-2-[4-(trifluoromethyl)-phenylmethyl-5-methylcyclopent-4,5]imidazo[2,1-b]purin-4(3H)one; furazlocillin; cis-2-hexyl-5-methyl-3,4,5,6a,7,8,9,9a-octahydrocyclopent[4,5]imridazo[2,1-b]purin-4-one; 3-acetyl-1-(2-chlorobenzyl)-2-propylindole-6-carboxylate; 4-bromo-5-(3-pyridylmethylamino)-6-(3-(4-chlorophenyl)propoxy)-3-(2H)pyridazinone; 1-methyl-5-(5-morpholinoacetyl-2-n-propoxyphenyl)-3-n-propyl-1,6-dihydro-7 H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); and Sch-51866.

Other type V phosphodiesterase inhibitors include, but are not limited to DMPPO (Eddahibi (1988) Br. J. Pharmacol., 125(4): 681-688), and 1-arylnaphthalene lignan series, including 1-(3-bromo-4,5-dimethoxyphenyl)-5-chloro-3-[4-(2-hydroxyethyl)-1-piperazinylcarbonyl]-2-(methoxycarbonyl)naphthalene hydrochloride (27q) (Ukita (1999) J. Med. Chem. 42(7): 1293-1305).

By “multi-organ systems” it is meant systems that affect multiple organs. Such organs include, but are not limited to, heart, liver, and brain.

By “nitric oxide donor” it is meant a compound that is able to donate nitric oxide or promote increase of nitric oxide. There are families of compounds that donate nitric oxide. Included among these compounds are: DETANONOate (DETANONO, NONOate or 1-substituted diazen-1-ium-1,2-diolates are compounds containing the [N(O)NO]— functional group: DEA/NO; SPER/NO; DETA/NO; OXI/NO; SULFI/NO; PAPA/NO; MAHMA/NO and DPTA/NO), PAPANONOate, SNAP (S-nitroso-N-acetylpenicillamine), sodium nitroprusside, and sodium nitroglycerine. There are compounds that promote the increase in nitric oxide, such as phosphodiesterase inhibitors and L-arginine.

By “promoting neurogenesis” as used herein, it is meant that neural growth is promoted or enhanced. This can include, but is not limited to, new neuronal growth or enhanced growth of existing neurons, as well as growth and proliferation of parenchymal cells and cells that promote tissue plasticity. Neurogenesis also encompasses, but is not limited to, neurite and dendritic extension and synaptogenesis.

By “augmentation” as used herein, it is meant that growth is either enhanced or suppressed as required in the specific situation. Therefore, if additional neuron growth is required, the addition of a nitric oxide donor increases this growth. Nitric oxide donors, or sources of nitric oxide, prime cerebral tissue to compensate for damage brought on by injury, neurodegeneration, or aging by enhancing receptor activation and promoting cellular morphological change and cellular proliferation.

By “neurological” or “cognitive” function as used herein, it is meant that the neural growth in the brain enhances the patient's ability to think, function, or more. Humans treated with nitric oxide have increased production of brain cells that facilitate improved cognition, memory and motor function. Further, patients suffering from neurological disease or injury when treated with nitric oxide have improved cognition, memory, and motor function.

The term “cell therapy” as used herein includes, but is not limited to, administering stem cells, a generalized mother cell whose descendants specialize into various cell types. The stem cells have various origins including, but not limited to, embryo, bone marrow, liver, stromal, fat tissue, and other stem cell origins known to those of skill in the art. These stem cells can be administered or placed into the desired areas as they naturally occur, or can be engineered in any manner known to those of skill in the art. Thus, through various genetic engineering methods including, but not limited to, transfection, deletion, and the like, the stem cells can be engineered in order to increase their likelihood of survival or for any other desired purpose.

The terms “enrich” or “enrichment” as used herein are meant to include, but are not limited to, to make rich or richer by the addition or increase of some desirable quality or quantity of substance. In the present invention, enrichment occurs by the addition or increase of more functional cardiac cells within or around the myocardium.

The terms “repopulate” or “repopulating” as used herein are meant to include, but are not limited to, the addition or replenishment of cardiac cells within or around the myocardium. These additionally reinforce the activity of currently functioning cells. Thus, replacement and/or reinforcement of existing cardiac cells occurs.

The purpose of the present invention is to promote an improved outcome from neuronal injury, or other injury, by augmenting the effects of the treatment, for example neurogenesis, and augmenting the cellular changes that promote functional improvement. For example, patients suffer neurological and functional deficits after stroke, CNS injury and neurodegenerative disease. These findings provide a means to enhance brain compensatory mechanism to improve function after CNS damage or degeneration. The induction of neurons and cellular changes induced by nitric oxide administration will promote functional improvement after stroke, injury, aging and degenerative disease. This approach can also provide benefit to patients suffering from other neurological disease such as, but not limited to, ALS, MS, and Huntington's disease. Additionally, the methods and compositions of the present invention can enhance the effectiveness of cell therapy.

Nitric oxide administered at propitious times after CNS injury promotes neurogenesis in brain and is able to facilitate neurogenesis. Nitric oxide can also augment the effectiveness of cell therapy. As an initial experiment, DETA/NO was employed, a compound with a long half-life (˜50 hours) which produces NO. Increased numbers of new neurons were identified when this compound was administered at and beyond 24 hours after onset of stroke. Preferably, the compounds of the present invention are administered directly to the site of injury. For example, the compounds can be administered orally, intraperitoneally, intravenously, or in any other manner known to those of skill in the art to provide the desired result. However, the compounds can be administered systemically if necessary for treatment.

The experimental data included herein show that a pharmacological intervention designed to induce production of NO can promote neurogenesis. Three compounds have been employed, DETANONOate, and sildenafil (Viagra™)) SNAP, these compounds have successfully induced neurogenesis and improved functional outcome after stroke. The compound used likely crosses the blood brain barrier. Neurogenesis is a major last goal in neuroscience research. Developing a way to promote production of neurons opens up the opportunity to treat a wide variety of neurological disease, CNS injury and neurodegeneration. It is possible to augment the production of neurons in non-damaged brain, so as to increase function.

The market for a class of drugs that promotes the production of neurons is vast. Nitric oxide donors, of which DETANONO is but one example, promote neurogenesis. Increasing neurogenesis translates into a method to increase, improve neurological, behavioral and cognitive function, with age and after injury or disease.

In recent years it has become abundantly clear that one mechanism for the deterioration of function in heart failure of any etiology is due, in part, to the ongoing death of heart muscle cells (Sabbah, 2000). The solution to this problem is to enrich or repopulate the myocardium with new cardiac cells, which take the place of lost cells or provide additional reinforcement of the currently function cardiac cells, thereby improving the pumping function of the failing heart. The present invention is based on the use of cells therapy to treat disease. Although stem cells have different origins (embryo, bone marrow, liver, fat tissue, etc.), their important common characteristic is that they have the potential to differentiate into various, if not all, cell types of the body. As previously mentioned, stem cells have been shown to be able to differentiate into cardiac muscle cells. (Maltsev et al., 1993 and 1994).

The present invention is advantageous over all currently existing treatments. For example, currently, treatment of heart failure is based primarily on the use of drugs that interfere with neurohumoral systems. Additionally, surgical treatment exists that include heart transplantation as well as the use of ventricular or bi-ventricular assisting devices. The advantages offered by the present invention is the ability to treat heart failure by directly addressing the primary cause of the disease, namely, loss of contractile units. Re-population of the myocardium with stem cells that differentiate into contractile units that contribute to the overall function of the failing heart, therefore, is novel and goes to the center of the problem. Other advantages include absence of side effects that are often associated with the use of pharmacological therapy and absence of immune rejection that plagues heart transplantation or other organ transplants.

The present invention has the potential to replace many current surgical therapies and possibly even pharmacological therapies. Devices currently exist that allow delivery of stem cells to the failing heart using catheter-based approaches, thus eliminating the need for open chest surgery. Additionally, the present invention is applicable in both the human medical environment and veterinary setting.

The present invention treats injury or disease and improves and/or restores normal function. More specifically, the present invention is used to augment cell therapy thereby enabling cell therapies to function more effectively and efficiently. Function is increased by enriching and/or repopulating the injured cells via transplanted stem cells that differentiate into the injured cells, thereby increasing function. Thus, the increase of contractile units increases the function of the heart. Additionally, the stem cells can also be responsible for the release of various substances such as trophic factors. Thus, for example, the release of trophic factors induces angiogenesis (increase of the number of blood vessels) in order to increase cardiac function and/or treat heart failure. Therefore, the stem cells operate to increase cardiac function and/or treat heart failure through various mechanisms other than just differentiating into functional cardiac muscle cells.

The general method of transplanting stem cells into the-myocardium occurs by the following procedure. The stem cells and the nitric oxide donor or PDE inhibitor are administered to the patient. The administration can be subcutaneously, parenterally including intravenous, intraarterial, intramuscular, intraperitoneally, and intranasal administration as well as with intrathecal and infusion techniques.

By the term “stem cell” is meant any form of cell therapy, including, but not limited to, hematopoietic cells which are capable of self-regeneration when provided to a human subject in vivo, and can become lineage restricted progenitors, which further differentiate and expand into specific lineages. As used herein, “stem cells” refers to hematopoietic cells and not stem cells of other cell types. Further, unless indicated otherwise, “stem cells” refers to human hematopoietic stem cells.

The term “stem cell” or “pluripotent” stem cell are used interchangeably to mean a stem cell having (1) the ability to give rise to progeny in all defined lineages, and (2) stem cells capable of fully reconstituting a seriously immunocompromised host in all blood cell types and their progeny, including the pluripotent hematopoietic stem cell, by self-renewal.

Bone marrow is the soft tissue occupying the medullary cavities of long bones, some haversian canals, and spaces between trabeculae of cancellous or spongy bone. Bone marrow is of two types: red, which is found in all bones in early life and in restricted locations in adulthood (i.e. in the spongy bone) and is concerned with the production of blood cells (i.e. hematopoiesis) and hemoglobin (thus, the red color); and yellow, which consists largely of fat cells (thus, the yellow color) and connective tissue.

As a whole, bone marrow is a complex tissue comprised of hematopoietic stem cells, red and white blood cells and their precursors, mesenchymal stem cells, stromal cells and their precursors, and a group of cells including fibroblasts, reticulocytes, adipocytes, and endothelial cells which form a connective tissue network called “stroma”. Cells from the stroma morphologically regulate the differentiation of hematopoietic cells through direct interaction via cell surface proteins and the secretion of growth factors and are involved in the foundation and support of the bone structure. Studies using animal models have suggested that bone marrow contains “pre-stromal” cells that have the capacity to differentiate into cartilage, bone, and other connective tissue cells. (Beresford, J. N.: Osteogenic Stem Cells and the Stromal System of Bone and Marrow, Clin. Orthop., 240:270, 1989). Recent evidence indicates that these cells, called pluripotent stromal stem cells or mesenchymal stem cells, have the ability to generate into several different types of cell lines (i.e. osteocytes, chondrocytes, adipocytes, etc.) upon activation. However, the mesenchymal stem cells are present in the tissue in very minute amounts with a wide variety of other cells (i.e. erythrocytes, platelets, neutrophils, lymphocytes, monocytes, eosinophils, basophils, adipocytes, etc.), and, in an inverse relationship with age, they are capable of differentiating into an assortment of connective tissues depending upon the influence of a number of bioactive factors.

As a result, the inventors have developed a process for isolating and purifying human mesenchymal stem cells from tissue prior to differentiation and then culture expanding the mesenchymal stem cells to produce a valuable tool for musculoskeletal therapy. The objective of such manipulation is to greatly increase the number of mesenchymal stem cells and to utilize these cells to redirect and/or reinforce the body's normal reparative capacity. The mesenchymal stem cells are harvested in great numbers and applied to areas of tissue damage to enhance or stimulate in vivo growth for regeneration and/or repair, to improve implant adhesion to various prosthetic devices through subsequent activation and differentiation, enhance hemopoietic cell production, etc.

Along these lines, various procedures are contemplated by the inventors for transferring, immobilizing, and activating the culture expanded, purified mesenchymal stem cells at the site for repair, implantation, etc., including injecting the cells at the site of a skeletal defect, incubating the cells with a prosthesis and implanting the prosthesis, etc. Thus, by isolating, purifying and greatly expanding the number of cells prior to differentiation and then actively controlling the differentiation process by virtue of their positioning at the site of tissue damage or by pretreating in vitro prior to their transplantation, the culture-expanded, undifferentiated mesenchymal stem cells can be utilized for various therapeutic purposes such as to elucidate cellular, molecular, and genetic disorders in a wide number of metabolic bone diseases, skeletal dysplasias, cartilage defects, ligament and tendon injuries and other musculoskeletal and connective tissue disorders.

Along these same lines, various procedures are contemplated by the inventors for transferring, immobilizing, and activating the mesenchymal stem or progenitor cells at the site for repair, implantation, etc., through the use of various porous ceramic vehicles or carriers, including injecting the cells into the location of injury.

The human mesenchymal stem cells can be obtained from a number of different sources, including plugs of femoral head cancellous bone pieces, obtained from patients with degenerative joint disease during hip or knee replacement surgery, and from aspirated marrow obtained from normal donors and oncology patients who have marrow harvested for future bone marrow transplantation. Although the harvested marrow was prepared for cell culture separation by a number of different mechanical isolation processes depending upon the source of the harvested marrow (i.e. the presence of bone chips, peripheral blood, etc.), the critical step involved in the isolation processes was the use of a specially prepared medium that contained agents which allowed for not only mesenchymal stem cell growth without differentiation, but also for the direct adherence of only the mesenchymal stem cells to the plastic or glass surface area of the culture dish. By producing a medium that allows for the selective attachment of the desired mesenchymal stem cells that were present in the marrow samples in very minute amounts, it was possible to separate the mesenchymal stem cells from the other cells (i.e. red and white blood cells, other differentiated mesenchymal cells, etc.) present in the bone marrow.

As indicated above, the complete medium can be utilized in a number of different isolation processes depending upon the specific type of initial harvesting processes used in order to prepare the harvested bone marrow for cell culture separation. In this regard, when plugs of cancellous bone marrow were utilized, the marrow was added to the complete medium and vortexed to form a dispersion which was then centrifuged to separate the marrow cells from bone pieces, etc. The marrow cells (consisting predominantly of red and white blood cells, and a very minute amount of mesenchymal stem cells, etc.) were then dissociated into single cells by passing the complete medium containing the marrow cells through syringes fitted with a series of 16, 18, and 20 gauge needles. It is believed that the advantage produced through the utilization of the mechanical separation process, as opposed to any enzymatic separation process, was that the mechanical process produced little cellular change while an enzymatic process could produce cellular damage particularly to the protein binding sites needed for culture adherence and selective separation, and/or to the protein sites needed for the production of monoclonal antibodies specific for said mesenchymal stem cells. The single cell suspension (which was made up of approximately 50-100.times.10.sup.6 nucleated cells) was then subsequently plated in 100 mm dishes for the purpose of selectively separating and/or isolating the mesenchymal stem cells from the remaining cells found in the suspension.

When aspirated marrow was utilized as the source of the human mesenchymal stem cells, the marrow stem cells (which contained little or no bone chips but a great deal of blood) were added to the complete medium and fractionated with Percoll (Sigma, St. Louis, Mo.) gradients more particularly described below in Example 1. The Percoll gradients separated a large percentage of the red blood cells and the mononucleate hematopoietic cells from the low density platelet fraction which contained the marrow-derived mesenchymal stem cells. In this regard, the platelet fraction, which contained approximately 30-50.times.10.sup.6 cells was made up of an undetermined amount of platelet cells, 30-50.times.10.sup.6 nucleated cells, and only about 50-500 mesenchymal stem cells depending upon the age of the marrow donor. The low-density platelet fraction was then plated in the Petri dish for selective separation based upon cell adherence.

In this regard, the marrow cells obtained from either the cancellous bone or iliac aspirate (i.e. the primary cultures) were grown in complete medium and allowed to adhere to the surface of the Petri dishes for one to seven days according to the conditions set forth in Example 1 below. Since no increase in cell attachment was observed after the third day, three days was chosen as the standard length of time at which the non-adherent cells were removed from the cultures by replacing the original complete medium with fresh complete medium. Subsequent medium changes were performed every four days until the culture dishes became confluent which normally required 14-21 days. This represented 10.sup.3-10.sup.4 fold increase in undifferentiated human mesenchymal stem cells.

The cells were then detached from the culture dishes utilizing a releasing agent such as trypsin with EDTA (ethylene diaminetetra-acetic acid) (0.25% trysin, 1 mM EDTA (1.times.), Gibco, Grand Island, N.Y.) or a chelating agent such as EGTA (ethylene glycol-bis-(2-amino ethyl ether) N,N′-tetraacetic acid, Sigma Chemical Co., St. Louis, Mo.). The advantage produced through the use of a chelating agent over trypsin was that trypsin could possibly cleave off a number of the binding proteins of the mesenchymal stem cells. Since these binding proteins contain recognition sites, when monoclonal antibodies were to-be produced, a chelating agent such as EGTA as opposed to trypsin, was utilized as the releasing agent. The releasing agent was then inactivated and the detached cultured undifferentiated mesenchymal stem cells were washed with complete medium for subsequent use.

These results indicated that under certain conditions, culture expanded mesenchymal stem cells have the ability to differentiate into bone when incubated as a graft in porous calcium phosphate ceramics. Although the internal factors which influence the mesenchymal stem cells to differentiate into bone as opposed to cartilage cells are not well known, it appears that the direct accessibility of the mesenchymal stem cells to growth and nutrient factors supplied by the vasculature in porous calcium phosphate ceramics, as opposed to the diffusion chamber, influenced the differentiation of the mesenchymal stem cells into bone.

As a result, the isolated and culture expanded mesenchymal stem cells can be utilized under certain specific conditions and/or under the influence of certain factors, to differentiate and produce the desired cell phenotype needed for tissue repair.

Administration of a single dose of mesenchymal stem cells can be effective to reduce or eliminate the T cell response to tissue allogeneic to the T cells or to “non-self” tissue, particularly in the case where the T lymphocytes retain their nonresponsive character (i.e., tolerance or anergy) to allogeneic cells after being separated from the mesenchymal stem cells.

The dosage of the mesenchymal stem cells varies within wide limits and is fitted to the individual requirements in each particular case. In general, in the case of parenteral administration, it is customary to administer from about 0.01 to about 5 million cells per kilogram of recipient body weight. The number of cells used will depend on the weight and condition of the recipient, the number of or frequency of administrations, and other variables known to those of skill in the art. The mesenchymal stem cells can be administered by a route that is suitable for the tissue, organ or cells to be transplanted. They can be administered systemically, i.e., parenterally, by intravenous injection or can be targeted to a particular tissue or organ, such as bone marrow. The human mesenchymal stem cells can be administered via a subcutaneous implantation of cells or by injection of stem cell into connective tissue, for example muscle.

The cells can be suspended in an appropriate diluent, at a concentration of from about 0.01 to about 5×10⁶ cells/ml. Suitable excipients for injection solutions are those that are biologically and physiologically compatible with the cells and with the recipient, such as buffered saline solution or other suitable excipients. The composition for administration must be formulated, produced and stored according to standard methods complying with proper sterility and stability.

Although the invention is not limited thereof, mesenchymal stem cells can be isolated, preferably from bone marrow, purified, and expanded in culture, i.e. in vitro, to obtain sufficient numbers of cells for use in the methods described herein. Mesenchymal stem cells, the formative pluripotent blast cells found in the bone, are normally present at very low frequencies in bone marrow (1:100,000) and other mesenchymal tissues. See, Caplan and Haynesworth, U.S. Pat. No. 5,486,359. Gene transduction of mesenchymal stem cells is disclosed in Gerson et al U.S. Pat. No. 5,591,625.

Unless otherwise stated, genetic manipulations are performed as described in Sambrook and Maniatis, MOLECULAR CLONING: A LABORATORY MANUAL, 2nd Ed.; Cold Spring Harbor Laboratory Press, Cold Spring Harbor, N.Y. (1989).

A detailed description of the method and composition of the present invention is set forth in Appendix A included herewith and incorporated by reference in its entirety. While specific embodiments are disclosed herein, they are not exhaustive and can include other suitable designs that vary in design and methodologies known to those of skill in the art. Basically, any differing design, methods, structures, and materials known to those skilled in the art can be utilized without departing from the spirit of the present invention.

Methods:

General methods in molecular biology: Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al, 1996, Blood 87:3822.)

General methods in immunology: Standard methods in immunology known in the art and not specifically described are generally followed as in Stites et al.(eds), Basic and Clinical Immunology (8th Edition), Appleton & Lange, Norwalk, Conn. (1994) and Mishell and Shiigi (eds), Selected Methods in Cellular Immunology, W.H. Freeman and Co., New York (1980).

Delivery of Therapeutics

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound or as pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein which treatment has a length proportional to the length of the disease process and drug effectiveness. The doses can be single doses or multiple doses over a period of several days, but single doses are preferred.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques which deliver it orally or intravenously and retain the biological activity are preferred.

In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered will vary for the patient being treated and will vary from about 100 ng/kg of body weight to 100 mg/kg of body weight per day and preferably will be from 10 mg/kg to 10 mg/kg per day.

### EXAMPLE 1

The effects of NO on angiogenesis and the synthesis of vascular endothelial growth factor (VEGF) were investigated in a model of focal embolic cerebral ischemia in the rat. Compared to control rats, systemic administration of an NO donor, DETANONOate, to rats 24 hours after stroke significantly enlarged vascular perimeters, and increased the number of proliferated cerebral endothelial cells and the numbers of newly generated vessels in the ischemic boundary regions, as evaluated by three-dimensional laser scanning confocal microscopy. Treatment with DETANONOate significantly increased VEGF levels in the ischemic boundary regions as measured by ELISA. A capillary-like tube formation assay was used to investigate whether DETANONOate increases angiogenesis in ischemic brain via activation of soluble guanylate cyclase. DETANONOate induced capillary-like tube formation was completely inhibited by a soluble guanylate cyclase inhibitor, 1H-[1,2,4]oxadiazolo[4,3-a]quinoxaline-1-one (ODQ). Blocking VEGF activity by a neutralized antibody against VEGF receptor 2 significantly attenuated DETANONOate-induced capillary-like tube formation. Moreover, systemic administration of a phosphodiesterase type 5 inhibitor (Sildenafil) to rats 24 hours after stroke significantly increased angiogenesis in the ischemic boundary regions. Sildenafil and an analog of cyclic guanosine monophosphate (cGMP) also induced capillary-like tube formation. These findings suggest that exogenous NO enhances angiogenesis in ischemic brain, which is mediated by the NO/cGMP pathway. Furthermore, the data suggest that NO, in part via VEGF, can enhance angiogenesis in ischemic brain.

Treatment of stroke with nitric oxide (NO) donors reduces functional neurological deficits. NO is a pleiotropic molecule that affects many physiological and pathophysiological functions. Animals treated with NO donors evoke cell proliferation in neurogenic regions of the brain, such as the subventricular zone and the dentate gyrus. However, the mechanisms underlying the improvement of neurological function after treatment require clarification.

A potential therapeutic target for NO treatment of stroke is angiogenesis. Administration of proangiogenic agents, such as basic fibroblast growth factor (bFGF) and vascular endothelial growth factor (VEGF), to animals with stroke significantly reduce neurological dysfunction. Inrcubation of human vascular smooth muscle cells with NO donors increases VEGF synthesis and the NO synthase (NOS) antagonist N^{W}nitro-I-arginine methyl ester (L-NAME) reduces VEGF generation. Endothelial NO synthase (eNOS) deficient mice exhibit significant impairment of angiogenesis in the ischemic limb, indicating that NO modulates angiogenesis in ischemic tissue. Thus, there appears to be a coupling between NO, VEGF and angiogenesis. However, there have been no studies on the effects of NO donors on VEGF and angiogenesis after stroke. Accordingly, the fact that NO increases VEGF and enhances angiogenesis via a cyclic guanosine monophosphate pathway (cGMP) was tested in a model of focal embolic cerebral ischemia in the rat.

Materials and Methods:

Animal Model:

Male Wistar rats weighing 320-380 gm were employed. The middle cerebral artery (MCA) was occluded by placement of an embolus at the origin of the MCA.

Experimental Protocol: 1) To examine whether exogenous NO affects neovascularization in ischemic animals, (Z)-1-[N-(2-aminoethyl)-N-(2-ammonioethyl)aminio]diazen-1-ium-1,2-diolate (DETANONOate), an NO donor-with a half-life of 57 hours under physiological conditions, was administered to ischemic rats. DETANONOate (0.4 mg/kg) was intravenously administered to rats (n=8) 24 hours after stroke and daily (i.p) for an additional 6 consecutive days. Ischemic rats (n=8) treated with the same volume of decayed DETANONOate were used as a control group. All rats were sacrificed 14 days after stroke. 2) To examine the effect of exogenous NO on brain levels of VEGF, DETANONOate (0.4 mg/kg) or saline was administered to ischemic rats (n=3 for each group) with the identical paradigm described in Protocol 1. These rats were sacrificed 7 days after stroke. 3) To examine whether increases in cGMP promote angiogenesis in ischemic brain, a phosphodiesterase type 5 (PDE 5) inhibitor which increases cGMP, Sildenafil dissolved in 3 ml of tap water (2 mg/kg), was fed to ischemic rats (n=8) at 24 hours after stroke and daily for an additional 6 days. Rats were sacrificed 14 days after stroke.

Bromodeoxyuridine Labeling:

Bromodeoxyuridine (BrdU, Sigma Chemical), the thymidine analog that is incorporated into the DNA of dividing cells during S-phase, was used for mitotic labeling. BrdU (50 mg/kg) was injected (i.p) daily for 13 consecutive days into ischemic rats starting 1 day after MCA occlusion.

Three Dimensional Image Acquisition and Analysis:

To examine neovascularization in ischemic brain, fluorescein isothiocyanate (FITC) dextran (2×10⁶ molecular weight, Sigma, St. Louis, Mo.; 0.1 ml of 50 mg/ml) was administered intravenously to the ischemic rats subjected to 14 days of MCAo. The brains were rapidly removed from the severed heads and placed in 4% of paraformaldehyde at 4° C. for 48 hours. Coronal sections (100 μm) were cut on a vibratome. The vibratome sections were analyzed with a Bio-Rad MRC 1024 (argon and krypton) laser-scanning confocal imaging system mounted onto a Zeiss microscope (Bio-Rad; Cambridge, Mass.), as previously described. Seven 100 μm thick vibratome coronal sections at 2 mm intervals from bregma 5.2 mm to bregma −8.8 mm from each animal injected with FITC-dextran were selected. Eight brain regions in the ipsilateral and contralateral hemispheres were selected within a reference coronal section (interaural 8.8 mm, bregma 0.8 mm). These regions were scanned in 512×512 pixel (276×276 μm²) format in the x-y direction using a 4× frame-scan average and twenty-five optical sections along the z-axis with a 1 μm step-size were acquired under a 40× objective. Vascular branch points, segment lengths and diameters were measured in three dimensions using software developed in the laboratory. Image acquisition and analysis were performed blindly.

Immunohistochemistry and Quantification:

For BrdU immunostaining, DNA was first denatured by incubating brain sections (6 μm) in 50% formamide 2×SSC at 65° C. for 2 hours and then in 2N HCl at 37° C. for 30 minutes. Sections were then rinsed with tris buffer and treated with 1% of H₂O₂ to block endogenous peroxidase. Sections were incubated with a mouse monoclonal antibody (mAb) against BrdU (1:1000, Boehringer Mannheim, Indianapolis, Ind.) overnight and incubated with biotinylated secondary antibody (1:200, Vector, Burlingame, Calif.) for 1 hour.

To quantify BrdU immunoreactive endothelial cells, numbers of endothelial cells and numbers of BrdU immunoreactive endothelial cells in ten enlarged vessels adjacent to the ischemic lesion were counted from each rat. Numbers of endothelial cells and BrdU immunoreactive endothelial cells in the ten vessels of the contralateral homologous area were also counted. Data are presented as percentage of BrdU immunoreactive endothelial cells to total endothelial cells in ten enlarged vessels from each rat.

Vascular perimeters were measured on coronal sections immunostained with an anti-von Willebrand factor antibody as previously described.

ELISA for VEGF:

The ischemic boundary regions and homologous tissue in the contralateral hemisphere were dissected. The tissue was homogenized and centrifuged at 10,000 g for 20 min at 4° C. and the supernatant was collected. ELISA for VEGF in the supernatants was performed using a commercially available kit specific for rat VEGF (R&D, systems) according to the manufacture's instruction.

Capillary-like Tube Formation Assay:

An in vitro angiogenesis assay was performed. Briefly, 0.8 ml of growth factor reduced Matrigel (Becton Dickinson) was added to pre-chilled 35 mm culture dishes and allowed to polymerize at 37° C. for 2 to 5 hours. Mouse brain-derived endothelial cells (2×10⁴ cells) were incubated for 3 hours in Dulbecco's modified Eagle's medium (DMEM) containing DETANONOate, Sildenafil, 1H-[1,2,4]oxadiazolo[4,3-a]quinoxaline-1-one (ODQ), 8-Br-cGMP, or a rat anti-mouse neutralizing antibody to VEGF receptor 2 (VEGFR2, DC101, Imclone System). For quantitative measurements of capillary tube formation, three random areas of Matrigel dishes were imaged and the length of continuous cords of three or more cells was measured.

Statistical Analysis:

One way analysis of variance (ANOVA) followed by Student-Newman-Keuls test was used. The data were presented as means±SE. A value of p<0.05 was taken as significant.

Results:

Effects of DETANONOate and Sildenafil on Angiogenesis In Vivo:

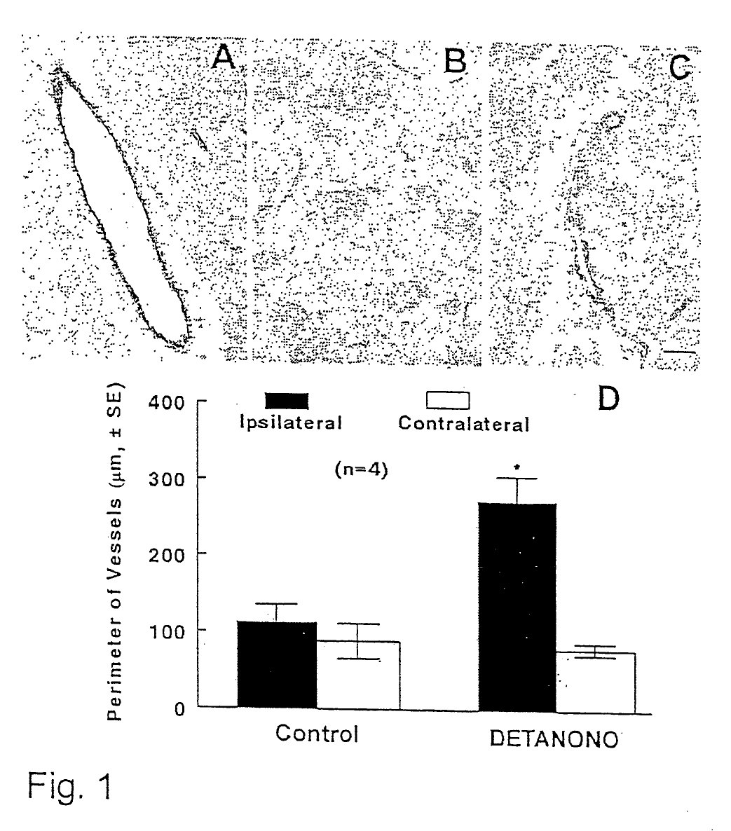

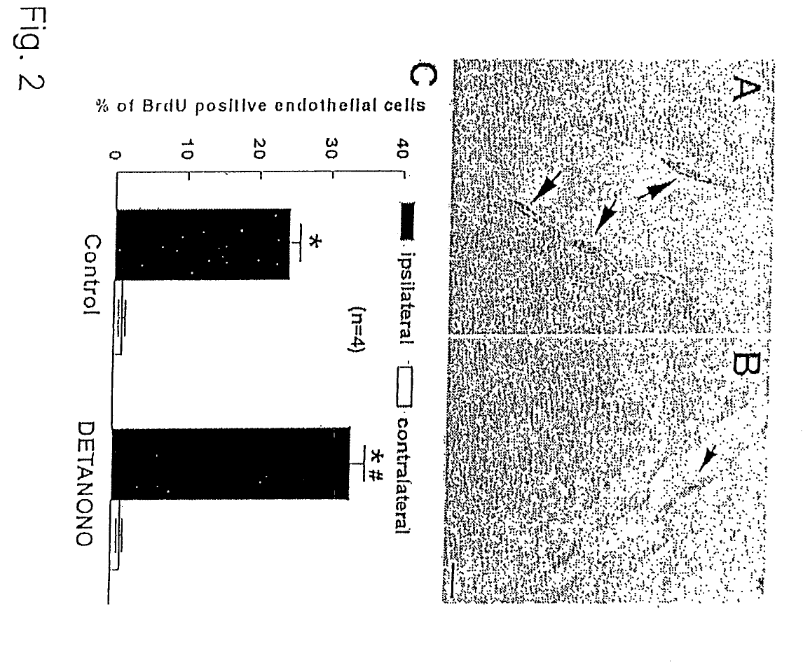

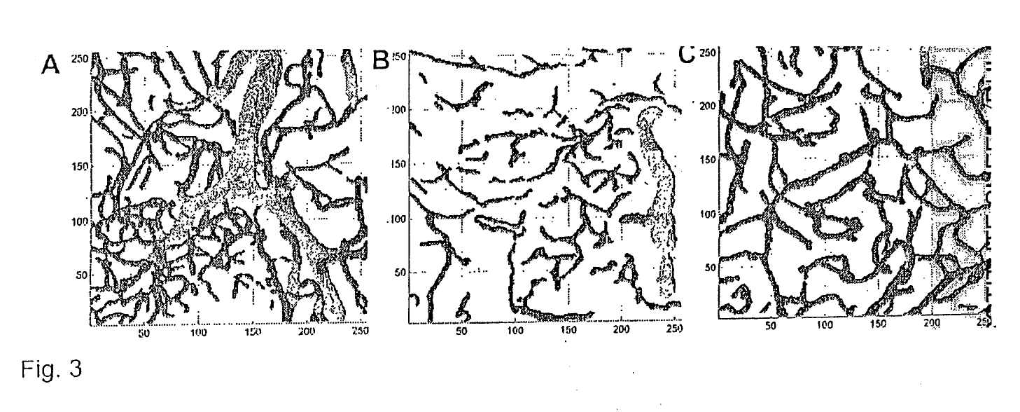

To examine whether exogenous NO enhances angiogenesis in ischemic brain, DETANONOate was administered to rats 24 hours after stroke for 7 days. Treatment with DETANONOate significantly (p<0.01) enlarged vascular perimeters (FIGS. 1A and 1D) around the ischemic lesion but did not enlarge vessels in the contralateral hemisphere (FIGS. 1B and 1D) compared with the ipsilateral vessels in the control rats (FIGS. 1C and 1D). Endothelial cells in enlarged thin walled vessels exhibited BrdU immunoreactivity (FIGS. 2A and 2B) and quantitative analysis revealed that the numbers of proliferated endothelial cells significantly (p<0.05) increased in rats treated with DETANONOate (FIG. 2C). To further examine angiogenesis, three dimensional analysis was performed using software developed in the laboratory, which measures numbers of segments, segment lengths and diameters of vessels. Treatment with DETANONOate significantly (p<0.05) increased the numbers of capillary segments in the boundary regions of ischemia (FIG. 3A and Table 1) compared with the numbers in ischemic rats treated with same volume of decayed DETANONOate (FIG. 3B and Table 1). The capillary segments in the DETANONOate treated groups exhibited significantly smaller diameters (FIG. 3A and Table 1) and shorter segment lengths (FIG. 3A and Table 1), suggesting that these are newly generated vessels. A significant increase of angiogenesis was also detected in rats treated sildenafil (Table 1).

Effects of DETANONOate and Sildenafil on Brain Levels of VEGF:

To examine whether administration of DETANONOate increases brain levels of VEGF, ELISA for endogenous rat VEGF was. performed. ELISA measurements revealed that treatment with DETANONOate significantly (p<0.05) increased VEGF levels in the ischemic boundary regions from 13.4±1.5 μg/ml in the control group (n=3) to 28.9±1.0 μg/ml in the DETANONOate treated group (n=3). Since NO increases cGMP, induction of VEGF by DETANONOate could occur via the cGMP pathway. PDE 5 is highly specific for hydrolysis of cGMP. Brain levels of VEGF in rats treated with the PDE 5 inhibitor, Sildenafil were measured. Treatment with Sildenafil significantly (p<0.05) increased VEGF levels (34.4±2.9 μg/ml vs 13.4±1.5 μg/ml in the control, n=3 per group) in the ischemic boundary.

Effects of Soluble Guanylate Cyclase Inhibitor and Neutralization of VEGFR2 on DETANONOate-induced Capillary-like Tube Formation:

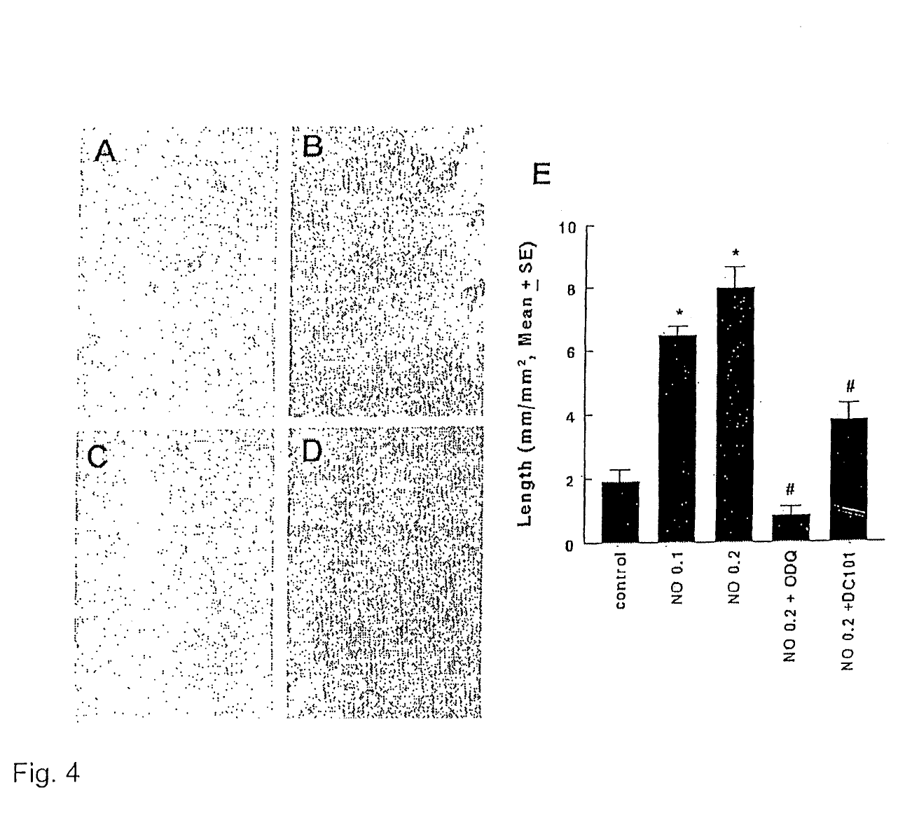

To support the hypothesis that DETANONOate increases angiogenesis in ischemic brain via the activation of soluble guanylate cyclase, the effects of DETANONOate on angiogenesis were further analyzed using a capillary-like tube formation assay. A significant increase in capillary-like tube formation was detected when mouse brain-derived endothelial cells were incubated with DETANONOate (0.2 μM, FIGS. 4B and 4E) compared with the endothelial cells incubated with DMEM only (FIGS. 4A and 4E). However, DETANONOate-induced capillary-like tube formation was completely inhibited when the endothelial cells were incubated with DETANONOate in the presence of ODQ, a potent inhibitor of soluble guanylate cyclase (FIGS. 4C and 4E), indicating that the NO/cGMP signaling pathway is involved in mediating the effects of DETANONOate on angiogenesis. To examine whether DETANONOate also enhances angiogenesis via increases in VEGF, the endothelial cells were incubated for 3 hours in the presence of DETANONOate (0.2 μM) and a rat anti-mouse neutralizing antibody to VEGFR2 (DC101, 10 μg/ml). The biological activity of this antibody against VEGFR2 in the mouse has been demonstrated. Treatment of endothelial cells with the antibody against VEGFR2 significantly (P<0.05) reduced DETANONOate-induced capillary-like tube formation (FIGS. 4D and 4E), suggesting that VEGF is involved in DETANONOate-induced angiogenesis.

Effects of Sildenafil on Capillary-like Tube Formation:

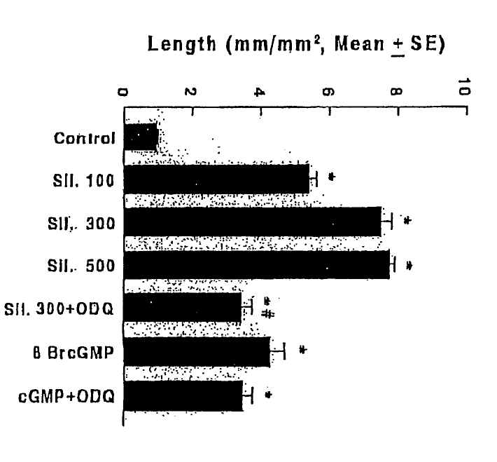

Incubation of the endothelial cells with Sildenafil (100 to 500 nM) produced concentration-dependent capillary-like tube formation (FIG. 5). 8-BrcGMP (1 mM), a stable analog of cGMP, also significantly (p<0.05) increased capillary-like tube formation (FIG. 5). ODQ (10 μM) significantly inhibited Sidenafil-induced capillary-like tube formation (FIG. 5), indicating that angiogenesis by Sildenafil is dependent on basal activity of sGC in the endothelial cells. ODQ did not significantly inhibit 8-BrcGMP-induced capillary-like tube formation (FIG. 5), confirming that this effect is independent of soluble guanylate cyclase activation.

Discussion:

The major findings of the present study are that 1) administration of DETANONOate or Sildenafil 24 hours after stroke increases synthesis of VEGF and enhances angiogenesis in ischemic brain; 2) ODQ, an inhibitor of soluble guanylate cyclase, completely inhibits DETANONOate-induced capillary-like tube formation; 3) Sildenafil, an inhibitor of PDE5, induces capillary-like tube formation; and 4) blocking of VEGF activity by a neutralized antibody against VEGFR2 attenuates DETANONOate-induced capillary-like tube formation; Together, these data indicate that exogenous NO enhances angiogenesis in ischemic brain via the NO/cGMP dependent pathway and an inhibitor of PDE 5 (Sildenafil) augments angiogenesis. The data also suggest a coupling of NO, VEGF and angiogenesis.

NO plays an important role in angiogenesis. However, there have been no studies on the effect of NO on angiogenesis in ischemic brain. Mice lacking eNOS exhibit severe impairment of spontaneous angiogenesis in response to limb ischemia, and administration of L-arginine accelerates angiogenesis. In the present study, administration of DETANONOate significantly increased the numbers of enlarged vessels and proliferated endothelial cells in the ischemic boundary regions, which is consistent with data that NO induces vessel dilation and endothelial cell proliferation.

NO activates soluble guanylate cyclase, thereby producing an increase of cGMP in target cells. PDE 5 enzyme is highly specific for hydrolysis of cGMP and Sildenafil citrate is a potent inhibitor of PDE5 which causes intracellular accumulation of cGMP 22. DETA/NONOate-induced capillary-like tube formation was completely inhibited by ODQ, a selective inhibitor of soluble guanylate cyclase, suggesting that DETA/NONOate enhances brain angiogenesis via activation of soluble guanylate cyclase. The results are in agreement with previous reports that NO activates soluble guanylate cyclase in angiogenesis. To obtain further evidence that increases in cGMP contribute to NO-enhanced angiogenesis in ischemic brain, the PDE 5 inhibitor (Sildenafil) was administered to rats 24 hours after stroke. The data show that treatment with Sildenafil enhances angiogenesis in the boundary regions of ischemia. Moreover, Sildenafil and 8-BrcGMP (an analog of cGMP) induce capillary-like tube formation in a culture of brain derived endothelial cells. ODQ significantly inhibits Sildenafil- but not 8-BrcGMP-induced capillary-like tube formation, indicating this response is dependent on basal activity of sGC. Therefore, the data support the conclusion that the NO/cGMP pathway mediates DETANONOate-induced angiogenesis in ischemic brain.

VEGF mediates angiogenesis and NO and VEGF can interact to promote angiogenesis. A high concentration of NO donor downregulates VEGF expression in endothelial cells. In contrast, recent studies show endogenous NO enhances VEGF synthesis. The eNOS-deficient mice exhibit significant impairment of angiogenesis in the ischemic hindlimb and administration of VEGF to these mice does not increase impaired angiogenesis, indicating that NO is a downstream mediator for VEGF-induced angiogenesis. Angiogenesis in response to VEGF depends on the tissue microenvironments. The data show that exogenous NO increased ischemic brain levels of VEGF and blocking VEGF activity attenuated DETANONOate-induced capillary-like tube formation, suggesting that NO induces VEGF synthesis in brain and VEGF at least in part mediates DETANONOate-induced angiogenesis. These findings are consistent with previous studies that NO derived from NO donors can increase the synthesis of VEGF. In addition, the PDE 5 inhibitor, Sildenafil, increases brain levels of VEGF in the ischemic brain, suggesting that cGMP likely contributes to NO-induced VEGF synthesis. This finding is inconsistent with a previous study that the cGMP is not involved in NO-induced upregulation of VEGF in cultured human articular chondrocytes. The reason for this discrepancy can be attributed to cell type difference, but remains enigmatic.

Angiogenesis is tightly regulated by two families of growth factors, the VEGF and angiopoietin families, as well as endothelial cell interaction with extracellular matrix. Upregulation of VEGF and angiopoietin genes are correlated with brain angiogenesis after stroke. Furthermore, stroke induces expression of VEGF receptors 1 and 2 in endothelial cells of cerebral vessels ¹². Administration of NO-donor could amplify endogenous VEGF in the astrocytes and endothelial cells and consequently increased VEGF enhances angiogenesis in ischemic brain via interaction with upregulated VEGF receptors in the endothelial cells, as previously demonstrated that treatment with VEGF increases angiogenesis in experimental stroke. Newly generated vessels function in ischemic brain, and they can contribute to functional recovery via improvement of long-term perfusion. Therefore, the positive interaction between NO and VEGF suggests that combination treatment with an NO donor and VEGF can have synergistic effects on angiogenesis.

FIG. 1 shows cerebral vascular perimeters. Treatment with DETANONOate enlarged cerebral vessels in the ischemic boundary (FIG. 1A), but not vessels in the homologous area of the contralateral hemisphere (FIG. 1B) from a representative rat. FIG. 1C shows an enlarged vessel in the ischemic boundary from a representative rat treated with decayed DETANONOate. Quantitative data (FIG. 1D) shows that treatment of stroke with DETANONOate significantly increased vascular perimeters compared with the ipsilateral vascular perimeters in the control rats. *p<0.01 vs ipsilateral. Bar in C=50 μm.

FIG. 2 shows proliferated cerebral endothelial cells. FIG. 2A shows several BrdU immunoreactive endothelial cells (arrows) in an enlarged thin-wall vessel of a representative rat treated with DETANONOate. FIG. 2B shows a BrdU immunoreactive endothelial cell (arrow) in an enlarged vessel of a representative rat from the control group. Although ischemia induced proliferation of endothelial cells (FIG. 2C, control), treatment with DETANONOate significantly increased the numbers of proliferated endothelial cells (FIG. 2C, DETANONO). *p<0.01 vs the contralateral hemisphere and #p<0.05 vs the ipsilateral hemisphere in the control group. Bar in B=10 μm.

FIG. 3 shows DETANONOate induces angiogenesis, as analyzed with three-dimensional images. Computer-generated images were originally derived from images obtained with three-dimensional laser scanning confocal microscopy. Treatment with DETANONOate increased the numbers of newly generated vessels (FIG. 3A), compared with the numbers of new vessels in rats in the control group (FIG. 3B). However, DETANONOate did not alter vascular morphology in the contralateral hemisphere (FIG. 3C). Green and red colors in the images represent vascular diameters larger and smaller than 7.5 μm, respectively. Image size is 276×276×25 μm³ and unit in the images is μm.

FIG. 4 shows DETANONOate induces in vitro angiogenesis. Mouse brain derived endothelial cells were incubated with DMEM for 3 hours in the absence of DETANONOate (FIG. 4A), in the presence of DETANONOate (0.2 μM, FIG. 4B), and in the presence of DETANONOate with ODQ (FIG. 4C) or with an antibody against VEGFR2 (FIG. 4D). Capillary-like tube formation was induced by DETANONOate (FIG. 4B), and this effect was inhibited by ODQ (FIG. 4C) or by the antibody against VEGFR2 (FIG. 4D). Similar results were obtained in at least four experiments. A bar graph (FIG. 4E) shows quantitative data of capillary-like tube formation. *p<0.05 vs control and #p<0.05 vs DETANONOate (0.2 μM). NO 0.1 and 0.2 represent DETANONOate 0.1 and 0.2 μM. DC101 represents the antibody against VEGFR2.

FIG. 5 shows a bar graph shows quantitative data of Sildenafil-induced capillary-like tube formation. Sildenafil (100-500 nM) and 8-BrcGMP induced capillary-like tube formation and ODQ significantly inhibited Sildenafil (300 nM) induced capillary-like tube formation but did not attenuate 8-BrcGMP-induced capillary-like tube formation. *p<0.05 vs control and #p<0.05 vs Sildenafil 300 nM. Sil.=Sildenafil.

### EXAMPLE 2

Methods

Male Wistar rats were subjected to embolic middle cerebral artery occlusion. Sildenafil (Viagra) was administered orally for 7 consecutive days starting 2 or 24 hours after stroke onset at doses of 2 or 5 mg/kg per day. Ischemic rats administered the same volume of tap water were used as a control group. Functional outcome tests (foot-fault, adhesive removal) were performed. Rats were killed 28 days after stroke for analysis of infarct volume and newly generated cells within the subventricular zone and the dentate gyrus. Brain cGMP levels, expression of PDE5, and localized cerebral blood flow were measured in additional rats.

Results

Treatment with sildenafil significantly (P=0.05) enhanced neurological recovery in all tests performed. There was no significant difference of infarct volume among the experimental groups. Treatment with sildenafil significantly (P=0.05) increased numbers of bromodeoxyuridine-immunoreactive cells in the subventricular zone and the dentate gyrus and numbers of immature neurons, as indicated by III-tubulin (TuJ1) immunoreactivity in the ipsilateral subventricular zone and striatum. The cortical levels of cGMP significantly increased after administration of sildenafil, and PDE5 mRNA was present in both nonischemic and ischemic brain.

Conclusions

Sildenafil increases brain levels of cGMP, evokes neurogenesis, and reduces neurological deficits when given to rats 2 or 24 hours after stroke. These data show that this drug that is presently in the clinic for sexual dysfunction has a role in promoting recovery from stroke.

Nitric oxide (NO) is a potent activator of soluble guanylate cyclase and causes cGMP formation in target cells. Phosphodiesterase type 5 (PDE5) enzyme is highly specific for hydrolysis of cGMP and is involved in regulation of cGMP signaling. Sildenafil is a novel inhibitor of PDE5 and causes intracellular accumulation of cGMP. Administration of an NO donor to rats with stroke significantly increases brain levels of cGMP, induces cell genesis, and improves functional recovery. Functional recovery is partly due to increases in levels of cGMP resulting from administration of an NO donor. Therefore, administration of sildenafil, a PDE5 inhibitor, to rats subjected to stroke enhances improvement of neurological outcome during stroke recovery.

Materials and Methods

Sildenafil is a weak basic compound, which is therefore only partially ionized at physiological pH and has a half-life of 0.4 hour in rats. A film tablet of Viagra (content 100 mg sildenafil, purchased commercially) was weighed and powdered.

Animal Model

Male Wistar rats weighing 320 to 380 g were used in the present study. The middle cerebral artery (MCA) was occluded by placement of an embolus at the origin of the MCA.

Experimental Protocols

The experiments were performed to examine whether administration of sildenafil affects cell proliferation and neurological behavior, sildenafil at 2 mg/kg (n=10) or 5 mg/kg (n=9) dissolved in 3 mL of tap water was administered orally to rats 2 hours after MCA occlusion and daily for an additional 6 days. Another group of the ischemic rats (n=10) was treated orally with sildenafil (2 mg/kg) 24 hours after MCA occlusion and daily for an additional 6. days. Ischemic rats (n=9) were treated with the same volume of tap water as a control group. Functional tests were performed and body weight was measured before ischemia and at 4, 7, 14, 21, and 28 days after onset of MCA occlusion. All rats were killed 28 days after MCA occlusion. Experiments were also performed to examine whether administration of sildenafil affects brain-cGMP levels, nonischemic rats were treated with sildenafil at 2 mg/kg (n=6) or tap water (n=10) for 7 days. These rats were killed 1 hour after the last treatment for measurements of brain levels of cGMP. Experiments were also performed to examine the effects of sildenafil on cerebral blood flow (CBF) and blood pressure, nonischemic rats (n=6) were treated orally with sildenafil, and local CBF and mean arterial blood pressure were measured starting at 30 minutes and continuing for 180 minutes after administration of sildenafil. Experiments were also performed to examine brain PDE5, nonischemic rats and ischemic rats were killed at 2, 4, 24, 48, and 168 hours after the onset of ischemia (n=3 for each time point). Reverse transcription (RT)-polymerase chain reaction (PCR) was performed to detect PDE5 in brain tissue.

cGMP Measurement in Brain Tissue

Levels of cGMP were measured with the use of a commercially available low-pH immunoassay kit (R&D Systems Inc). The sensitivity of the assay was approximately 0.6 μmol/mL for the nonacetylated procedure. The brain was rapidly removed, and the cortex and the cerebellum were separated. Brain tissue was weighed and homogenized in 10 volumes of 0.1N HCl containing 1 mmol/L 3-isobutyl-1-methylxanthine.

RT-PCR Analysis

To examine the presence of PDE5 in rat brain tissue, primers for PDE5A1 and PDE5A2 were synthesized according to published sequence. The 5′ primer 5′-AAAACTCGAGCAGAAACCCGCGGCA-AACACC-3′ and the 3′ primer 5′-GCATGAGGACTTTGAG-GCAGAGAGC-3′ amplified a cDNA fragment coding for N-terminal regions of rat PDE5A1. The 5′ primer 5′-ACCTCTGCTATGTTGCCCTTTGC-3′ and the 3′ primer 5′-GCATGAGGACTTTGAGGCAGAGAGC-3′ amplified a cDNA fragment coding to rat PDE5A2. For cDNA synthesis, total RNA extracted from brain tissue was reverse transcribed. Samples were denatured at 95° C. for 2 minutes and then amplified for 40 cycles. Each cycle consisted of denaturation at 95° C. for 30 seconds, annealing at 62° C. for 1 minute, and extension at 72° C. for 2 minutes. The samples (30 μL per well) were electrophoresed on 1.5% agarose containing ethidium bromide.

Body Weight Loss

Animals were weighed before and at 4, 7, 14, 21, and 28 days after embolic ischemia. Body weight loss is presented as a percentage of preischemic body weight.

Foot-Fault Test

Rats were tested for placement dysfunction of forelimbs with the modified foot-fault test before ischemia and at 4, 7, 14, 21, and 28 days after embolic ischemia. Rats were set on an elevated hexagonal grid of different sizes and placed their paws on the wire while moving along the grid. With each weight-bearing step, the paw may fall or slip between the wire. The total number of steps (movement of each forelimb) that the rat used to cross the grid was counted, and the total number of foot-faults for each forelimb was recorded.

Adhesive Removal Test

An adhesive removal test was used to measure somatosensory deficits and was performed before MCA occlusion and at 4, 7, 14, 21 and 28 days after MCA occlusion.

Bromodeoxyuridine Labeling

Bromodeoxyuridine (BrdU) was used to measure cell proliferation. Animals received daily intraperitoneal injections of BrdU (50 mg/kg; Sigma) on the day of stroke and subsequently for 14 consecutive days. Cell proliferation in the subventricular zone and dentate gyrus was measured in rats killed at 28 days (in experimental protocol 1, all 4 groups) after ischemia.

Immunohistochemistry

For BrdU immunostaining, DNA was first denatured by incubating brain sections (6 m) in 50% formamide 2′ SSC at 65° C. for 2 hours and then in 2N HCl at 37° C. for 30 minutes. Sections were then rinsed with Tris buffer and treated with 1% of H2 O2 to block endogenous peroxidase. Sections were incubated with a primary antibody to BrdU (1:100) at room temperature for 1 hour and then incubated with biotinylated secondary antibody (1:200, Vector) for 1 hour. Reaction product was detected with the use of 3′3′-diaminobenzidine-tetrahydrochloride (DAB; Sigma). For 'III-tubulin (TuJ1) immunostaining, which identifies immature neurons, 12 coronal sections were incubated with the antibody against TuJ1 (1:1000) at 4° C. overnight and were then incubated with a biotinylated horse anti-mouse immunoglobulin antibody at room temperature for 30 minutes. Double immunofluorescent staining for BrdU and TuJ1 was performed to determine whether BrdU-immunoreactive cells express neuronal phenotype on the coronal sections.

Image Analysis and Quantification

Measurements of BrdU-immunoreactive cells were performed on paraffin-embedded 6-4m-thick sections.11 BrdU-immunostained sections were digitized with the use of a 40 objective (Olympus BX40) via the MCID computer imaging analysis system (Imaging Research). BrdU-immunoreactive nuclei were counted on a computer monitor to improve visualization and in 1 focal plane to avoid oversampling. All BrdU-immunoreactive-positive nuclei were counted in both the ipsilateral and contralateral walls of the lateral ventricle of the subventricular zone and in the dentate gyrus. For the subventricular zone, every 40th coronal section was selected from each rat for a total of 7 sections between anteroposterior 10.6 mm of the genu corpus callosum and anteroposterior 8.74 mm of the anterior commissure crossing. For the dentate gyrus, every. 50th coronal section was selected from each rat for a total of 8 sections between anteroposterior 5.86 mm and anteroposterior 2.96 mm of the granule cell layer. BrdU-immunoreactive nuclei in the subventricular zone and in the dentate gyrus are presented as the number of the cells per square millimeter (mean SE). Density values for the 7 sections (subventricular zone) and 8 sections (dentate gyrus) were averaged to obtain a mean density value for each animal. Numbers of TuJ1-immunoreactive cells were counted in the subventricular zone and striatum, and data are presented as the number of TuJ1-immunoreactive cells per section (mean SE).

Monitoring of Relative Erythrocyte Flow Velocity

Relative erythrocyte flow velocity was measured by laser-Doppler flowmetry (PeriFlux PF4 flowmeter; Perimed AB) in the tissue under the laser-Doppler flowmetry probe.13 A burr hole 1.5 mm in diameter was drawn on the skull 2 mm posterior to the bregma and 6 mm lateral to midline.13 The dura was left intact. After the application of mineral oil onto the burr hole, the probe was placed 0.5 mm above the dural surface. Relative flow velocities were measured 30 minutes after administration of sildenafil. This measurement reflects relatively localized CBF.14 Values of flow velocities are presented as a percentage of the contralateral hemispheric values.

Measurements of Infarct Volume

Measurement of infarct volume was measured on 7 hematoxylin and eosin-stained coronal sections with the use of a Global Laboratory Image analysis program (Data Translation). Briefly, the area of both hemispheres and the infarct area (mm 2) were calculated by tracing the area on the computer screen. Infarct volume (mm 3) was determined by multiplying the appropriate area by the section interval thickness. The infarct volume is presented as the percent-age of infarct volume of the contralateral hemisphere (indirect volume calculation).

Statistical Analysis

For analysis of neurological functional recovery and body weight, the generalized estimation equations (GEE) analysis approach was used instead of ANOVA because the data did not meet assumptions of normality and equal variance for ANOVA. A paired t test or signed rank test was used to test the difference in cell proliferation between ipsilateral and contralateral regions of subventricular zone, dentate gyrus, and striatum. The GEE analysis approach was used to study the treatment effect on cell proliferation in the ipsilateral and contralateral subventricular zone regions, dentate gyrus, and striatum. All values are presented as mean SE. Statistical significance was set at P 0.05.

Results

Effects of Sildenafil on Cell Proliferation

Ischemic rats treated with sildenafil (2 or 5 mg/kg) initiated at 2 or 24 hours after stroke had significant (P 0.05) increases in numbers of BrdU-immunoreactive cells in the dentate gyrus of both hemispheres (Table 1) compared with control rats. Treatment with sildenafil at a dose of 2 mg/kg (at 2 or 24 hours) significantly (P 0.05) increased numbers of BrdU-immunoreactive cells in the ipsilateral subventricular zone (Table 1), and the 5 mg/kg dose (at 2 hours) significantly (P 0.05) increased numbers of BrdU-immunoreactive cells in the subventricular zone of both hemispheres (Table 1) compared with numbers of BrdU-immunoreactive cells in control rats.

FIG. 6 shows the effect of treatment with sildenafil increased TuJ1-immunoreactive cells 28 days after ischemia. FIG. 6A is a sample from a representative rat, robust increases in numbers of TuJ1-immunoreactive cells in the ipsilateral subventricular zone compared with the contralateral subventricular zone (FIG. 6B) are shown. Ependymal cells (arrows in FIGS. 6A and B) were not TuJ1 immunoreactive. TuJ1-immunoreactive cells exhibited cluster in the ipsilateral striatum (FIG. 6C) compared with the homologous tissue in the contralateral hemisphere (FIG. 6D). Double immunostaining with anti-bodies against TuJ1 and BrdU shows that BrdU-immunoreactive cells (FIGS. 6E and G, green, arrows) were TuJ1 immunoreactive (FIGS. 6E and F, red, arrows). FIG. 6E is a merged image from FIGS. 6F and G. FIGS. 6H and I show quantitative data of numbers of TuJ1-immunoreactive cells in the subventricular zone (n 6 in each group) and striatum (n 6 in each group), respectively. (*P=0.05, **P=0.01, #P=0.05 vs control group. LV indicates lateral ventricle. Bars 10 m in FIGS. 1B and G and 20 m in C.)

TABLE 1
Density of Newborn Cells in Brain

| | Subventricular Zone | | Dentate Gyrus | |
|---|---|---|---|---|
| Groups | Ipsilateral | Contralateral | Ipsilateral | Contralateral |
| Sildenafil 2 mg/kg, 2 h | 383 ± 23.44* | 296 ± 19.74 | 55 ± 3.99* | 55 ± 2.10† |
| Sildenafil 5 mg/kg, 2 h | 437 ± 32.97† | 312 ± 23.79* | 59 ± 5.26* | 58 ± 5.38* |
| Sildenafil 2 mg/kg, 24 h | 374 ± 16.07* | 295 ± 24.54 | 57 ± 4.21* | 56 ± 4.76* |
| Control | 295 ± 32.69 | 246 ± 18.54 | 44 ± 2.96 | 42 ± 3.01 |

Density of newborn cells is presented as mean ± SEM number of BrdU-immunoreactive cells per mm².
*P < 0.05,
†P < 0.01 vs control group.

Effects of Sildenafil on Immature Neurons

Administration of sildenafil robustly increased number of TuJ1-immunoreactive cells in the ipsilateral subventricular zone (FIG. 6A) and striatum (FIG. 6C). TuJ1-immunoreactive cells exhibited clusters in the ipsilateral striatum (FIG. 6C). Some of the TuJ1-immunoreactive cells were BrdU immunoreactive (FIGS. 6E to 6G). Quantitative measurements revealed that administration of sildenafil at a dose of 2 or 5 mg/kg significantly (P=0.05) increased numbers of TuJ1-immunoreactive cells in the ipsilateral and contralateral subventricular zones compared with the number in control rats (FIG. 6H). Treatment with sildenafil also significantly increased the number of TuJ1 cells in the ipsilateral striatum compared with homologous tissue in the contralateral hemisphere and in the ipsilateral striatum of control rats (FIG. 6I).

Effects of Sildenafil on Neurological Outcome

The ischemic rats treated with sildenafil at a dose of 2 or 5 mg/kg significantly improved performance on the foot-fault test (Table 2) and the adhesive removal test (Table 3) during 4 to 21 days compared with control rats when treatment was initiated at 2 hours after onset of ischemia. In addition, treatment with sildenafil at doses of 2 and 5 mg/kg significantly reduced animal body weight loss (Table 4). In contrast, infarct volumes measured 28 days after ischemia were not significantly different among these groups (Table 5), suggesting that infarct volume does not contribute to improvement of functional recovery. Sildenafil was also administered at a dose of 2 mg/kg to the ischemic rats starting at 24 hours after onset of ischemia. Ischemic rats receiving sildenafil exhibited significant (P=0.05) improvements at the foot-fault (Table 2) and adhesive removal (Table 3) tests during 7 to 28 days after stroke. Rats treated with sildenafil also showed a significant (=0.05) reduction in body weight loss at 4, 7, 14, 21, and 28 days after ischemia (Table 4). However, there were no significant differences in infarct volume between ischemic animals treated with sildenafil and animals in the control group (Table 5).

Effects of Sildenafil on cGMP

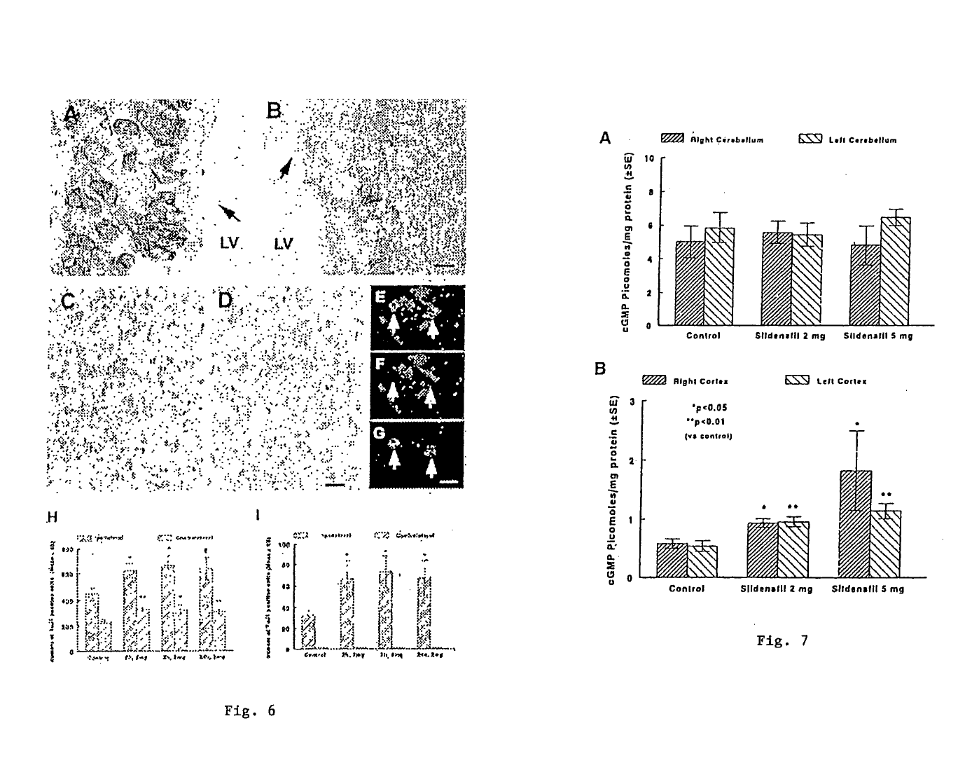

The cerebellar levels of cGMP (FIG. 7A, control) were higher than the cortical (FIG. 7B, control) levels in nonischemic control rats, which is consistent with previous studies. 4 Treatment with sildenafil at a dose of 2 or 5 mg/kg for 7 days significantly (P=0.05) increased the cortical levels of cGMP (FIG. 7B) compared with levels in the control group.

Effects of Sildenafil on Localized CBF

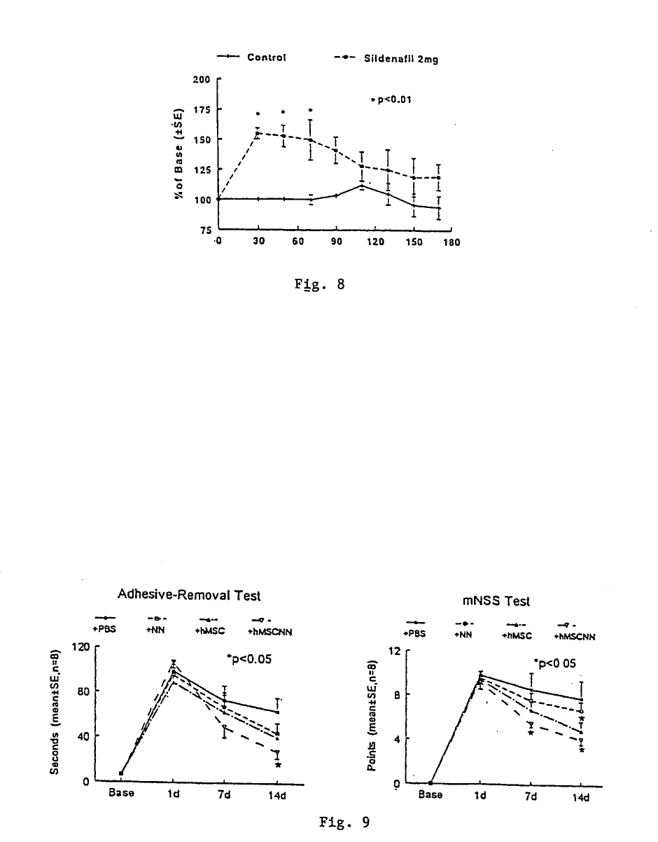

Administration of sildenafil at a dose of 2 mg/kg to nonischemic rats significantly increased localized CBF levels compared with the control rats (FIG. 8). Significantly increased localized CBF persisted for 70 minutes after administration of sildenafil (FIG. 8).

PDE5 in Rat Brain

RT-PCR analysis revealed both PDE5A1 (257 bp) and PDE5A2 (149 bp) transcripts in non-ischemic rat brain tissue, indicating the presence of PDE5 (data not shown). Levels of PDE5A1 and PDE5A2 mRNA measured by band density (n=3 for each time point) did not show a statistical difference after MCA occlusion compared with the nonischemic rats.

Discussion

The present study demonstrates that treatment of focal cerebral ischemia in rats with sildenafil significantly improved recovery of neurological outcome and significantly increased numbers of BrdU- and TuJ1-immunoreactive cells in ischemic brain. In addition, administration of sildenafil significantly increased cortical levels of cGMP. Therefore, the data show that increased cGMP levels resulting from administration of sildenafil mediates enhanced neurological outcome.

TABLE 2
Foot-Fault Test

| | % of Foot-Faults | | | | | |
|---|---|---|---|---|---|---|
| Groups | Before Ischemia | 4 d | 7 d | 14 d | 21 d | 28 d |
| Sildenafil 2 mg/kg, 2 h | 1.1 ± 0.01 | 22.81 ± 3.1 | 15.2 ± 1.6† | 13.2 ± 1.2† | 9.1 ± 1.5† | 8.2 ± 1.4 |
| Sildenafil 5 mg/kg, 2 h | 1.02 ± 0.02 | 17.9 ± 2.9 | 16.6 ± 1.4* | 14.6 ± 2.1* | 9.5 ± 1.6† | 7.6 ± 1.4 |
| Sildenafil 2 mg/kg, 24 h | 1.03 ± 0.03 | 25.3 ± 3.8 | 14.4 ± 1.0† | 10.0 ± 0.5† | 9.0 ± 0.5† | 5.3 ± 0.8* |
| Control | 1.06 ± 0.07 | 31.4 ± 3.4 | 24.9 ± 3.0 | 22.0 ± 2.6 | 19.4 ± 2.7 | 11.8 ± 1.9 |

Values are mean ± SE for specified number of days after ischemia.
*P < 0.05,
†P < 0.01 vs control group.

TABLE 3
Adhesive Removal Test

| | Seconds | | | | | |
|---|---|---|---|---|---|---|
| Groups | Before Ischemia | 4 d | 7 d | 14 d | 21 d | 28 d |
| Sildenafil 2 mg/kg, 2 h | 7.0 ± 0.1 | 96.4 ± 9.8 | 41.9 ± 8.4† | 27.6 ± 3.9† | 23.6 ± 5.1* | 15.9 ± 3.8 |
| Sildenafil 5 mg/kg, 2 h | 16.7 ± 0.4 | 100.7 ± 9.2 | 70.7 ± 10.8* | 38.6 ± 7.9† | 26.0 ± 6.6† | 14.8 ± 3.9 |
| Sildenafil 2 mg/kg, 24 h | 6.8 ± 0.3 | 102 ± 6.6 | 49.4 ± 4.5† | 14.1 ± 1.3† | 14.0 ± 1.0† | 10.7 ± 0.9 |
| Control | 7.0 ± 0.3 | 114.7 ± 3.6 | 95.7 ± 5.2 | 67.8 ± 9.6 | 43.4 ± 5.7 | 19.0 ± 3.3 |

Values are mean ± SE.
*P < 0.05,
†P < 0.01 vs control group.

PDE5 is an important enzyme for the hydrolysis of cGMP. The observations of PDE5 mRNA in the cortex in nonischemic rats are consistent with previous studies in which PDE5 mRNA and proteins were detected in rats. Sildenafil citrate is a potent inhibitor of PDE5 and causes intracellular accumulation of cGMP. The data show that administration of sildenafil significantly increased brain cGMP levels. in parallel with the findings, local administration of zaprinast, a relatively selective inhibitor of PDE5, to rat brain slices leads to an increase of cGMP release. Thus, the data indicate that sildenafil affects brain PDE5. cGMP modulates vasorelaxing effects in vascular muscle. Administration of sildenafil transiently increased CBF in nonischemic rats, consistent with previous in vitro and in vivo studies. Administration of zaprinast elicits dilatation of the basilar artery in rats and produces dilatation of dog cerebral arteries. Administration of sildenafil at a dose of 5-mg/kg decreases the systolic arterial blood pressure, and the effect lasts for at least 6 hours. However, the effects of sildenafil on CBF do not provide neuroprotection because the treatment did not reduce infarct volume and the treatment was effective even when sildenafil was first administered at 24 hours after the onset of ischemia, which is far beyond the therapeutic window for neuroprotection.

Another new finding of the present study is that treatment with sildenafil significantly increases proliferation of progenitor cells in the subventricular zone and the dentate gyrus and numbers of immature neurons, as assayed by TuJ1 immunostaining. Administration of DETA/NONOate, an NO donor, significantly enhances neurogenesis. NO activates soluble guanylate cyclase and leads to formation of cGMP, while sildenafil inhibits PDE5 activity and results in inhibition of cGMP breakdown. Taken together, these data show that cGMP regulates neurogenesis. The findings are consistent with previous studies that cGMP-dependent protein kinase type I enhances sensory neuron precursor proliferation. It is interesting to note that neuronal progenitor cells in the subventricular zone migrate to the olfactory bulb, and after reaching the olfactory bulb, they differentiate into mature neurons. These data are consistent with the observation that formation of olfactory memory is mediated by cGMP concentration. cGMP levels in neurons are also involved in the modulation of dendritic and axonal guidance. Increased intracellular cGMP via sema can convert dendritic and axonal guidance from repulsion to attraction. In addition, cGMP enhances neurite outgrowth in hippocampal neurons in culture and in PC12 cells. Furthermore, aged rats exhibit a decrease in the basal levels of cGMP as a consequence of a more active degradation of cGMP by a phosphodiesterase in the aged brain compared with the adult brain. Decreases in NO and cGMP synthesis in aged brain can have important functional implications in the processes of learning and memory. Neurogenesis can translate into functional improvement. For example, mice with a high rate of neurogenesis in the dentate gyrus exhibit enhanced performance on a hippocampal-dependent task, whereas a de-creasing rate of neurogenesis is correlated with impairment on such a task. Therefore, enhancement of neurogenesis can contribute to functional recovery after treatment with sildenafil. In summary, the results of this study demonstrate that administration of sildenafil after stroke enhances functional recovery and augments neurogenesis in the rat.

FIG. 7 shows that levels of cGMP in the cerebellum (FIG. 7A) and cortex (FIG. 7B) after treatment with sildenafil in nonischemic rats (n=6); n=10 in control group.

TABLE 4
Animal Body Weight Loss

| | % of Preischemic Body Weight | | | | | |
|---|---|---|---|---|---|---|
| Groups | Before Ischemia | 4 d | 7 d | 14 d | 21 d | 28 d |
| Sildenafil 2 mg/kg, 2 h | 100 | 81.8 ± 2.3 | 86.6 ± 2.5† | 89.6 ± 4.7* | 98.2 ± 3.7† | 105.2 ± 3.0* |
| Sildenafil 5 mg/kg, 2 h | 100 | 84.6 ± 1.8 | 81.8 ± 2.5* | 84.7 ± 3.9 | 95.2 ± 3.5* | 101.3 ± 4.3 |
| Sildenafil 2 mg/kg, 24 h | 100 | 86.9 ± 0.2 | 84.9 ± 1.8† | 89.2 ± 2.6* | 99.5 ± 2.0* | 107.2 ± 2.5* |
| Control | 100 | 74.7 ± 1.1 | 73.8 ± 1.1 | 77.6 ± 3.4 | 81.5 ± 4.7 | 92.4 ± 4.6 |

Values are mean ± SE.
*P < 0.05,
†P < 0.01 vs control group.

TABLE 5
Infarct Volume

| Groups | Infarct Volume, % |
|---|---|
| Sildenafil 2 mg/kg, 2 h | 35.2 ± 3.3 |
| Sildenafil 5 mg/kg, 2 h | 37.7 ± 4.3 |
| Sildenafil 2 mg/kg, 24 h | 35.5 ± 0.9 |
| Control | 38.3 ± 1.7 |

Infarct volume is presented as mean ± SE percentage of lesion relative to the contralateral hemisphere.

### EXAMPLE 3

Male Wistar rats (n=32) were subjected to middle cerebral artery occlusion (MCAo) and were randomized with 8 rats in four treatment groups, with treatment initiated 1 day after stroke. Groups included: 1) phosphate buffered saline (PBS); 2) subtherapeutic DETA-NONOate (NN) at a dose of 0.4 mg/kg (IP); 3) subtherapeutic hMSCs (1×10⁶ cells-iv); and 4) combination subtherapeutic NN and hMSCs. Functional outcome measurements consisted of a Neurologic Severity Scale (18 point scale) (NSS) and the Adhesive Removal Test performed prior to stroke, immediately before treatment and at 7 and 14 days after treatment. Data were well balanced among groups before the treatment (p-values>0.30). hMSC by NONO interaction was observed at 1.4 days (p-value=0.86). However, there was an overall hMSC effect on NSS at 14 days. Rats with the treatment of hMSC+NONO had a significant improvement on NSS at 14 days compared to rats in the control group (p-value=0.01), while rats in the low dose hMSC had a borderline improvement on NSS at 14 days (pvalue=0.05) compared to rats in the control. No significant difference on NSS at 14 days was detected between the control and NONO treated groups (p=0.64) and between the hMSC treated and the hMSC+NONO treated groups (p=0.48). The same treatment effects were observed on Adhesive-Removal test score at 14 days; rats treated with the hMSC+NONO had a significant improvement at 14 days compared to rats in the control group (p-value=0.01). There was a borderline improvement at 14 days in rats treated with the low dose (i.e. subtherapeutic) of hMSC alone and no significant improvement in rats treated with subtherapeutic NONO alone compared to the rats in controls with p-values of 0.06 and 0.64 respectively. At 7 days, the neurological functional improvement was observed only on NSS for the rats treated with the combination of hMSC and NONO compared to rats in the control group (p-value=0.03). These data indicate that combination of subtherapeutic therapeutic modalities of hMSCs and an NO donor (DETA-NONOate) significantly improves functional outcome compared to control PBS treatment animals.

Volume of Cerebral Infarction and the Presence of MSCs in Ischemic Brain:

No significant reduction of volume of ischemic damage was detected in rats with hMSC (30.7±6.2%) or NONOate (32.2±6.2%) and combination hMSCs with NONOate (28.7±6.7%) treatment, compared with control rats subjected to MCAo with PBS (34.9±7.4%). hMSCs were identified immunohistochemically using an antibody specific for human chromosomes (MAB1281). Within the brain tissue, cells derived from hMSCs were characterized by MAB1281 staining. No MAB1281 positive cells were found in the non-hMSCs treated rats. MSCs identified by MAB1281 survived and were distributed throughout the damaged brain of recipient rats. MAB1281 positive cells were observed in multiple areas, including cortex and striatum of the ipsilateral hemisphere. The vast majority of MAB1281 positive hMSCs were located in the ischemic boundary zone. Few cells were observed in contralateral hemisphere. There was no significant increase in numbers of MAB1281 cells between the hMSC and combination therapy groups. These data indicate that the volume of cerebral infarction is not affected by the combination therapy and that the numbers of MSCs that enter brain is not altered by the coadministration of an NO donor.

Neurogenesis:

BrdU (50 mg/kg-ip) was injected daily for 14 days after treatment in all groups. BrdU is a thymidine analog that labels newly formed DNA and thereby identifies newly formed cells. FIG. 9 shows that in the ipsilateral hemisphere subventricular zone, BrdU positive cells were significantly increased in the hMSC (2b, 40.6±10.7) or/and NONOate (FIG. 9c, 43.6±10.0/section; FIG. 9d, 67.4±22.8/section) treated group compared to the control PBS treatment group (FIG. 9a, 29.8±8.8/section) (p<0.05). BrdU found in the cytoplasm of macrophage-like cells were not counted. Double staining shows that the BrdU positive cells express the neuronal markers NeuN, neuron specific enolase (NSE) and the astrocyte marker GFAP. The percentage of BrdU reactive cells that express NeuN and GFAP proteins was approximately, 3%, 3% and 6%, respectively. These data indicate that that while individual subtherapeutic NO donor and MSC therapy failed to significantly increase neurogenesis compared to PSC control treated animals, combination therapy significantly promotes neurogenesis in ischemic brain.

Angiogenesis:

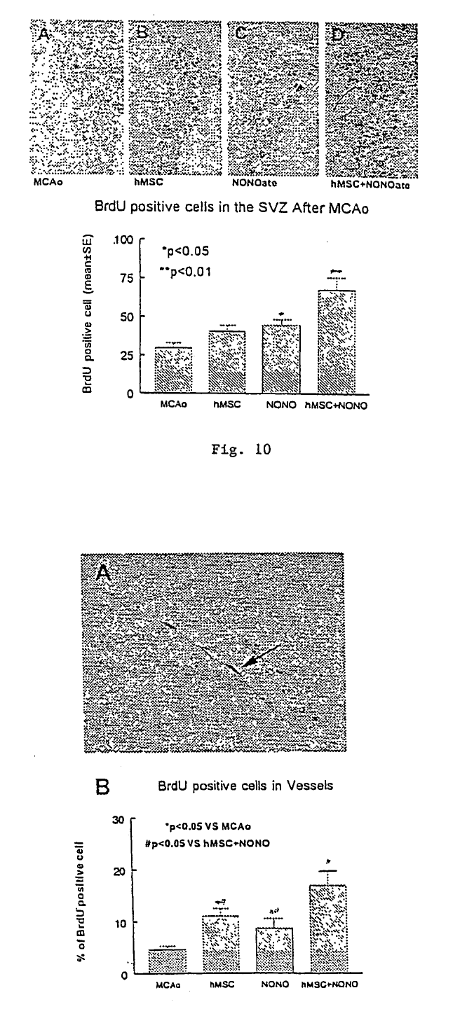

Enlarged and thin walled vessels are termed “mother” vessels and have been found under conditions of cerebral ischemic angiogenesis. FIG. 10 shows that enlarged vessels exhibited a significant (p<0.05) increase in BrdU immunoreactive endothelial cells (FIG. 10a) in hMSCs treatment group and NONOate treatment groups compared with control MCAO group in the ipsilateral hemisphere. BrdU reactive endothelial cells were significantly increased in the ipsilateral hemisphere of the combination subtherapeutic hMSCs/NONOate treatment group compared with the ipsilateral hemisphere of hMSCs or NONOate alone treatment groups (FIG. 10b, p<0.05). These data indicate that combination NO donor and MSC therapy significantly increases angiogenesis compared with the individual therapies.

Enhanced angiogenesis after combination therapy is also demonstrated in FIG. 11 which shows three-dimensional images of cerebral vessels in the ischemic penumbra after MCAO following the treatment 1) PBS; 2) NONOate; 3) hMSCs; 4) hMSCs+NONOate. FIG. 11A shows original composite images of FITC-dextran perfused cerebral microvessels. FIGS. 11B and C are computer generated three-dimensional images derived from the original images. Different colors in FIG. 11B represent individual vessels, which were not connected to each other. Green and red colors in FIG. 11C code for diameter of blood vessels less then 7.5 μm (red) and larger then 7.5 μm (green), respectively. Three-dimensional quantitative data revealed that hMSCs with or without NONOate treatment significantly (p<0.05) increased numbers of branch points in the penumbra compared with numbers found in the ipsilateral hemisphere of rats subjected to control MCAo. Segments of capillaries were significantly (p<0.05) shorter in the ipsilateral hemisphere of the hMSC or/and NONOate treated group and the PBS control group than in the homologous tissue in the contralateral hemisphere, indicating that these are newly formed vessels after stroke in ipsilateral hemisphere. Vascular diameter in the ipsilateral penumbra after hMSCs treatment significantly (p<0.05) increased compared with the homologous area of the contraiateral hemisphere and control MCAo animals. Enlarged vessels can develop into capillaries after ischemia. Vessel surface area significantly (p<0.05) increased in hMSC with or without NONOate treated animals compared with control MCAO animals in the ipsilateral hemisphere. Taken together, these data demonstrate that hMSCs with or without NONOate treatment enhances angiogenesis in the ischemic brain. These data complement the BrdU angiogenesis data and indicate that combination therapy promotes angiogenesis.

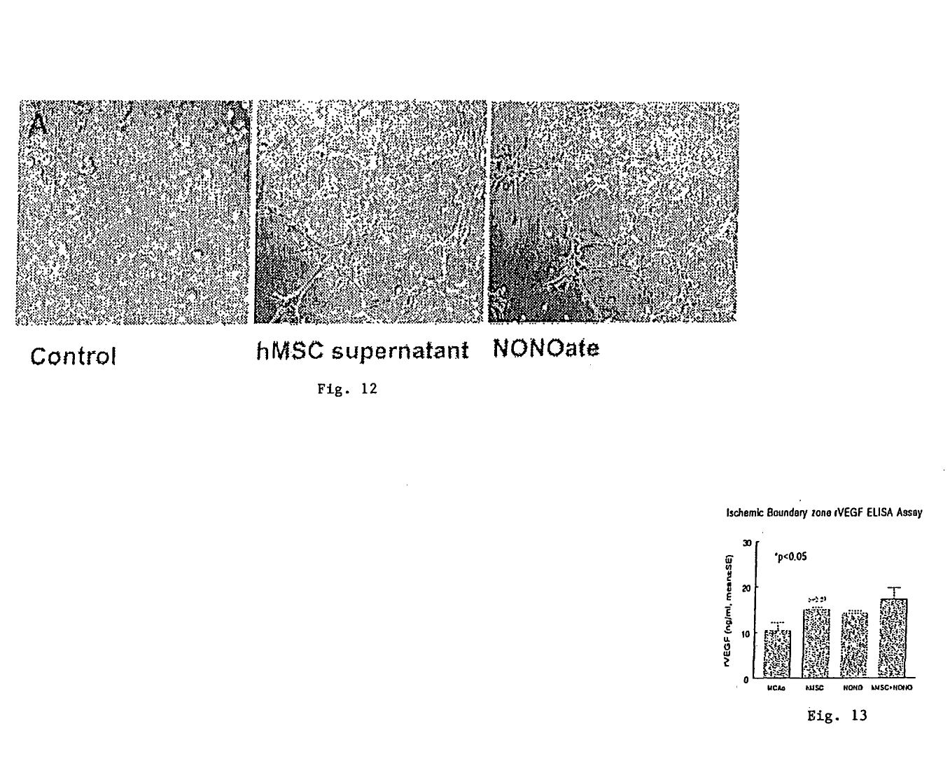

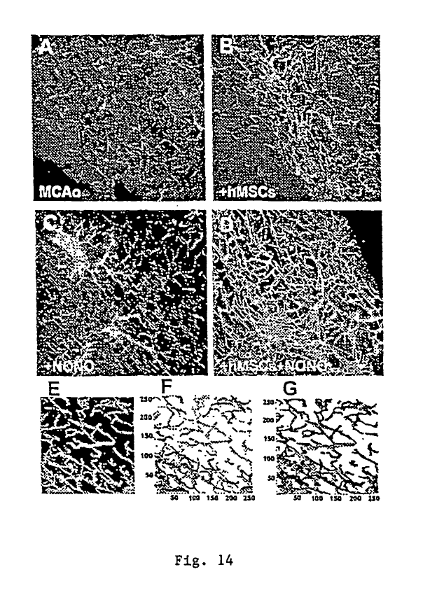

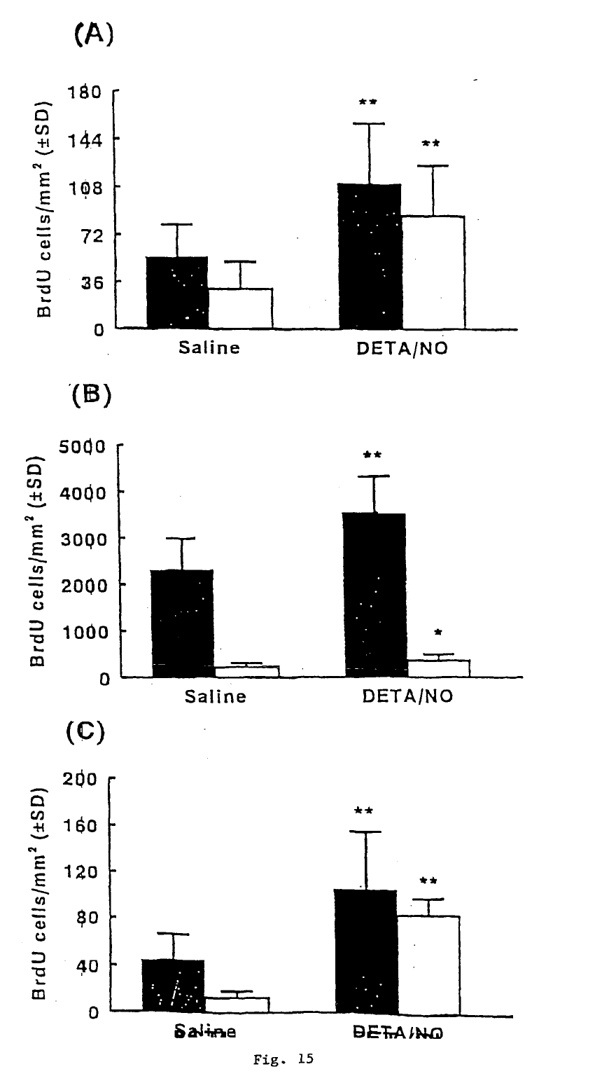

The enhance induction of angiogenesis is also evident from in vitro studies of tubule formation in brain derived endothelial cells. FIG. 12 demonstrates that hMSC-supernatant (FIG. 12b) and NONOate (FIG. 12c) strongly induces endothelial tube formation by brain-derived endothelial cells compared with control medium (DMEM, FIG. 12a). The endothelial cells formed a network of capillary-like structures with numerous intercellular contacts. Total tube length was significantly increased (p<0.01) in supernatant from cultural hMSCs (6.9±0.72 mm/mm²) and NONOate treatment (4.6±0.6 mm/mm²) compared with the control medium (DMEM, 1.4 [symbol] 0.1 mm/mm ). Total tube length was significantly increased in supernatant from cultural hMSCs compared with the NONOate. These data show that both hMSCs and NONOate promote capillary-tube formation.

VEGF:

To obtain insight into the mechanisms associated with the induction of angiogenesis and neurogenesis, the fact that combination therapy induces the expression of neurotrophic and growth factors in brain was tested. Data are presented on the levels of vascular endothelial growth factor (VEGF) in brain after treatment with MSCs, DETA-NONOate, combination (MSC+NONO) therapy, and control, PBS treated animals subjected to MCAo. FIG. 13 shows that using the Sandwich ELISA method, VEGF secretion from endogenous cells (rat VEGF) was significantly increased in hMSCs with NONOate treatment groups compared with MCAo control group. Rat VEGF secretion was borderline increased in hMSCs alone treatment group. Single dose NONOate alone treatment did not show a significant increase in VEGF compared with MCAo control group. These data indicate that subtherapeutic combination MSC+NONO therapy significantly enhances VEGF secretion compared to individual therapy.

### EXAMPLE 4

Induction of Cell Proliferation in Normal Monischemic Animals:

The effects of an NO donor administered to normal young adult rats on the induction of cell proliferation within three regions of brain, the dentate gyrus, the olfactory bulb (OB) and the subventricular zone (SVZ) were tested. An NO donor, (Z)-1-[N-(2-aminoethyl)-N-(2-ammonioethyl)aminio]diazen-1-ium-1,2-diolate (DETA/NONO-ate), was selected, because this compound is a highly efficient NO donor with a half-life of 57 hours under physiological conditions (Beckman, 1995; Estevez et al., 1998). Young male Wister rats (3-4 month old) received 4 consecutive I.V bolus doses of DETA/NONOate (0.1 mg/kg each, every 15 min, and total dose of 0.4 mg/kg) on the first experimental day and DETA/NONOate (0.4 mg/kg) was administered (i.p) daily for an additional 6 consecutive days. Rats that received saline were used as a control group. Bromodeoxyuridine (BrdU) was used as mitotic labeling to measure cell proliferation. Animals received daily i.p injections of BrdU (50 mg/kg, Sigma) on the first experimental day and subsequently for 14 consecutive days. Rats were sacrificed at 14 and 42 days after the treatment. BrdU immunoreactive nuclei were counted on a computer monitor to improve visualization and in one focal plane to avoid over-sampling. Structures were sampled either by selecting predetermined areas on each section (OB) or by analyzing entire structures on each section (SVZ and dentate gyrus) (Zhang et al., 2001). All BrdU immunoreactive-positive nuclei in these areas are presented as the number of the BrdU immunoreactive cells/mm2. Density for the selected several sections was averaged to obtain a mean density value for each animal (Zhang et al., 2001).

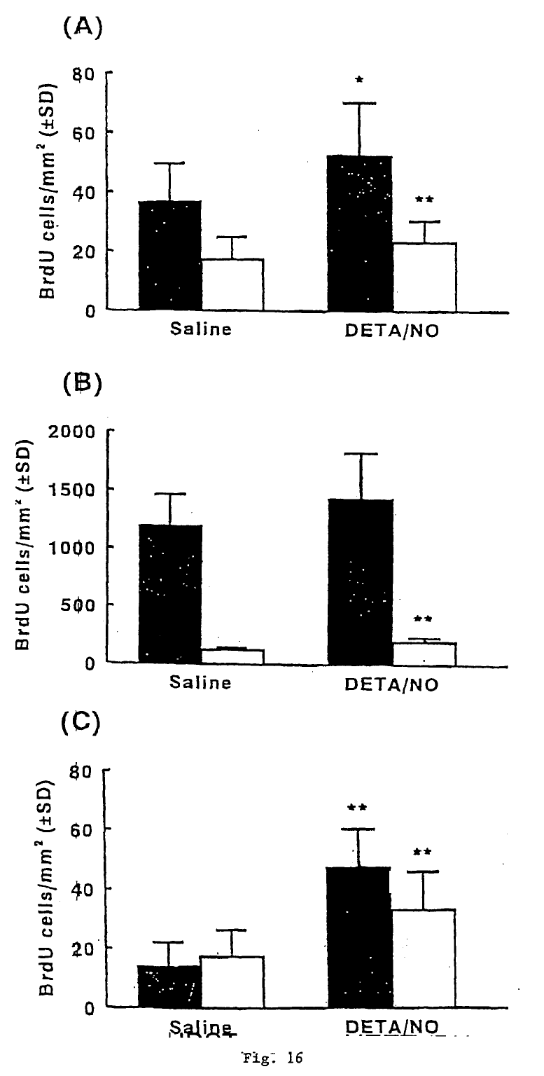

FIG. 15 shows the cell proliferation in the brain of the young adult rats, administered DETA/NONO-ate. Statistically significant increased in the numbers of BrdU reactive cells were demonstrated within the dentate gyrus (FIG. 15A), SVZ (FIG. 15B) and OB (FIG. 15C). More than 95% of the newly generated cells within the dentate gyrus exhibited neuronal markers of NeuN and MAP2, indicating that these cells have the potential to integrate into the tissue. The cells within the SVZ and the OB were not characterized with double-labeled immunohistochemistry. However, morphologically, they resembled proliferating cells. The progenitor cells in the SVZ of the lateral ventricle migrate into the OB (Alvarez-Buylla et al., 2000). Thus, these data clearly indicate that NO, which in the developing brain has been associated with cell proliferation and migration, induces cell proliferation and migration in the adult brain. In these studies only cell proliferation was measured in both groups of animals, and not the behavioral and functional effects of cell proliferation. However, there are substantial supporting data that cell proliferation within the dentate gyrus translates into improved learning in mice (Gould and Gross, 2002).

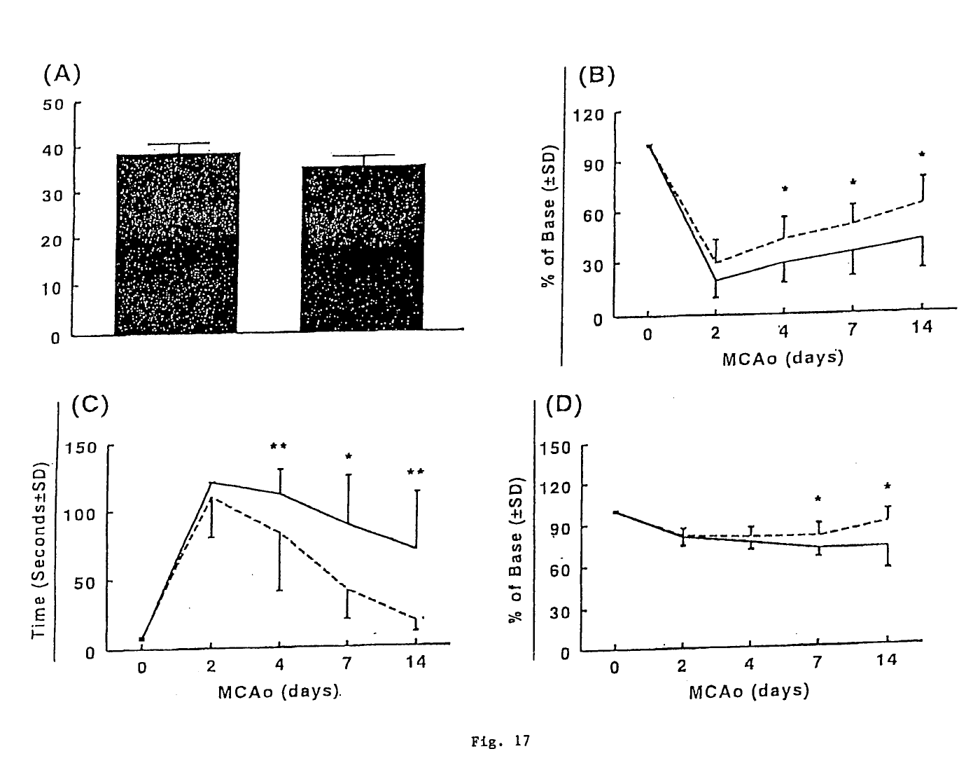

The question of whether this induction of neurogenesis by means of an NO donor in young adult (3-4 month old) rats would also be present in older rats was also tested. To test this hypothesis, 18-month-old male Wistar rats were treated with DETA/NONOate, using the identical experimental protocol described for young rats. FIG. 16 shows cell proliferation the three regions, dentate gyrus (FIG. 16A), SVZ (FIG. 16B), OB (FIG. 16C), and described above for the young rats. As in the young rats, treatment with DETA/NONOate significantly increased the number of proliferating cells. For the saline treated animals, the baseline cell proliferation was reduced by approximately a factor of 2 in the SVZ and dentate gyrus. In the SVZ and dentate gyrus, treatment with DETA/NONOate increased cell proliferation in a similar ratio in the old as well as the young animals. The relative increase in the number of proliferated cells within the OB was not as robust in the old animals as in the young animals. This may be attributed to a loss of cell migration potential in the old compared to the young animals.

These data provide novel and important observations. One is that cell proliferation can be induced in the old animals as in the young animals. The percent increase in proliferation is similar for the old and the young animals. However, very obvious, is the decease in the absolute numbers of proliferating cell in the old versus the young rats. Functional correlates of cell proliferation in the old animals were not measured, and consequently we have no data on whether the increase in cells within the dentate translates into improved function.

NO Donor Enhances Functional Recovery After Stroke:

It has been demonstrated that treatment of DETA/NONOate induces cell proliferation and neurogenesis in non-ischemic young rats as well as in young rats subjected to embolic stroke (Zhang et al., 2001). The treatment of rats initiated at one day after stroke translated into significant functional benefit. Thus, the data demonstrate that a pharmacological agent that releases NO when administered to animals 1 day after induction of a major ischemic stroke encompassing the territory of the middle cerebral artery (MCA) improves functional outcome (Zhang et al., 2001).

The question arises as to the specificity of this NO agent for the induction of neurogenesis and functional benefit. Are the effects specific for DETA/NONOate or would other agents that donate NO likewise provide functional benefit? To test this question, rats were treated with another NO donor, S-nitroso-N-acetylpenicillamine (SNAP, Sigma) that is structurally different from DETA/NONOate. Young male adult Wistar rats were subjected to embolic MCA occlusion (Zhang et al., 1997). SNAP at a dose of 30 μg/kg was intravenously administered to rats as a bolus followed by 300 μg/kg/h infusion for 60 minutes at 24 hours after embolic MCA occlusion. As functional outcome measures, the rotarod test which assesses motor function such as coordination and balance (Zhang et al., 2000), the adhesive removal test which measures forelimb somato-sensorimotor asymmetries (Schallert et al., 2000), and animal body weight were measured prior to treatment and at 2, 4, 7, and 14 days after treatment. Animals were sacrificed at 14 days after stroke and the infarct volume was measured (Zhang et al., 1997). FIG. 17 shows infarct volume and the functional outcome measurements for the saline treated and the SNAP treated groups. There was no significant difference in volume of cerebral infarction between the treated and the non-control treated groups (FIG. 17A).. However, significant improvement in function measured with the rotarod (FIG. 17B) and the adhesive removal test (FIG. 17C) was noted by 4 days after the onset of stroke. These benefits persisted to the time of sacrifice at 14 days post stroke. Animal body weight (FIG. 17D), as an index of general physiological well being, was significantly increased compared to vehicle-saline treated animals at 7 days after stroke. These data clearly demonstrate that treatment with an NO donor such as SNAP provides significant functional benefit to the animals, without affecting the volume of cerebral infarction (FIG. 17A). Thus, the effect of the treatment is one of restorative therapy not neuroprotective therapy. These data indicate that pharmacological agents, such as NO donors, can enhance function after stroke. Functional improvement in these animals is associated with change and remodeling of brain. Neurogenesis and cell proliferation as well as angiogenesis and increased levels of synaptic proteins are induced by the NO donor molecules.

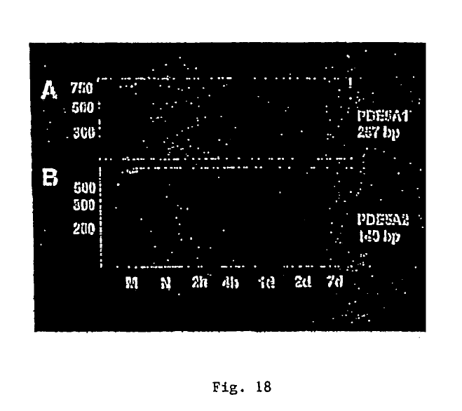

NO is an activator of soluble guanylate cyclase and causes increased cGMP in target cells (Ignarro, 1989; Garthwaite and Boulton, 1995). cGMP has been associated with changes in axon extension and modification of neuronal connections (Williams et al., 1994). It is possible that cGMP itself plays an important role in promoting brain plasticity. Increase brain levels of cGMP in rats treated with NO donors indicate that the NO donors enter the brain (Zhang et al., 2001). Another way to induce an increase in cGMP in brain is to inhibit the activity of the enzyme that breaks down cGMP. Phosphodiesterase type 5 (PDE 5) enzyme is highly specific for hydrolysis of cGMP (Corbin and Francis, 1999; Kotera et al., 2000). One way therefore to reduce the breakdown of cGMP and hence to increase levels of cGMP in brain is to reduce or inhibit PDE 5. To test the effect of administering a compound that inhibits PDE 5, adult male rats were fed sildenafil (2 mg/kg) daily for 7 days at 24 hours after the onset of stroke. FIG. 18 shows the presence of PDE 5 in brain. Feeding the animals sildenafil significantly improved functional outcome, as measured in an array of functional outcome measurements (Zhang et al., 2002). This therapeutic benefit was evident without a reduction of cerebral infarction, a similar condition observed with other NO donors. Thus, these data show that cGMP can be an important mediator of brain plasticity after stroke. This plasticity can also improve functional response.

In general, these data indicate that agents that affect NO and cGMP can alter normal, aged and injured brain. Not only is cell proliferation and angiogenesis increased, but also, significant functional benefit is obtained. There are other, cellular based ways to induce brain remodeling and functional improvement after stroke and neural injury. One approach, which has clinical implications, is to employ a population of cells, such as bone marrow stromal cells. These cells when administered to rodents enter brain and evoke the production of a variety of neurotrophic factors and cytokines that remodel brain and provide significant functional benefit (Review, Chopp and Li, 2002).

FIG. 15 includes bar graphs that show the number of BrdU immunoreactive cells in the dentate gyrus (FIG. 15A), in the SVZ (FIG. 15B), and in the OB (FIG. 15C) in non-ischemic young adult rats at 14 ( [symbol] ) and 42 ( [symbol] ) days after treatment with DETA/NONOate or saline. *p<0.05 and **p<0.01 versus the saline treated group.

FIG. 16 includes bar graphs that show the number of BrdU immunoreactive cells in the dentate gyrus (FIG. 16A), in the SVZ (FIG. 16B), and in the OB (FIG. 16C) in non-ischemic aged rats at 14 ( [symbol] ) and 42 ( [symbol] ) days after treatment with DETA/NONOate or saline. *p<0.05 and **p<0.01 versus the saline treated group.

FIG. 17 shows the effect of SNAP treatment on infarct volume (FIG. 17A), rotarod (FIG. 17B) and adhesive removal (FIG. 17C) tests as well as animal body weight (FIG. 17D). *p<0.05 and **p<0.01 versus the saline treated group. n=8 for each group.

FIG. 18 shows RT-PCR of PDE5A1 (FIG. 18A) and PDE5A2 (FIG. 18B) mRNA in the cortex of non-ischemic rats (N in FIG. 18A and FIG. 18B) and the ipsilateral cortex of rats 2 hours to 7 days after ischemia. M=marker, N=non-ischemic rats, 2 hours, 4 hours, 1 day, 2days and 7days =times after ischemia.

Conclusion:

It has been demonstrated that a pharmacological therapy based on NO and cGMP induces changes in brain that enhance restoration of function after stroke, and induce cell proliferation and neurogenesis in the normal young and old animal. These data along, with other studies on promotion of brain plasticity using cell-based therapy, opens new opportunities to treat neurodegenerative disease and neural injury.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the described invention, the invention may be practiced otherwise than as specifically described.

References

Burke and Olson, “Preparation of Clone Libraries in Yeast Artificial-Chromosome Vectors” in Methods in Enzymology, Vol. 194, “Guide to Yeast Genetics and Molecular Biology”, eds. C. Guthrie and G. Fink, Academic Press, Inc., Chap. 17, pp. 251-270 (1991).
Capecchi, “Altering the genome by homologous recombination” Science 244:1288-1292 (1989).
Davies et al., “Targeted alterations in yeast artificial chromosomes for inter-species gene transfer”, Nucleic Acids Research, Vol. 20, No. 11, pp. 2693-2698 (1992).
Dickinson et al., “High frequency gene targeting using insertional vectors”, Human Molecular Genetics, Vol. 2, No. 8, pp. 1299-1302 (1993).
Duff and Lincoln, “Insertion of a pathogenic mutation into a yeast artificial chromosome containing the human APP gene and expression in ES cells”, Research Advances in Alzhimer's Disease and Related Disorders, 1995.
Huxley et al., “The human HPRT gene on a yeast artificial chromosome is functional when transferred to mouse cells by cell fusion”, Genomics, 9:742-750 (1991).
Jakobovits et al., “Germ-line transmission and expression of a human-derived yeast artificial chromosome”, Nature, Vol. 362, pp. 255-261 (1993).
Lamb et al., “Introduction and expression of the 400 kilobase precursor amyloid protein gene in transgenic mice”, Nature Genetics, Vol. 5, pp. 22-29 (1993).
Pearson and Choi, Expression of the human b-amyloid precursor protein gene from a yeast artificial chromosome in transgenic mice. Proc. Natl. Acad. Sci. USA, 1993. 90:10578-82:
Rothstein, “Targeting, disruption, replacement, and allele rescue: integrative DNA transformation in yeast” in Methods in Enzymology, Vol. 194, “Guide to Yeast Genetics and Molecular Biology”, eds. C. Guthrie and G. Fink, Academic Press, Inc., Chap. 19, pp. 281-301 (1991).
Schedl et al., “A yeast artificial chromosome covering the tyrosinase gene confers copy number-dependent expression in transgenic mice”, Nature, Vol. 362, pp. 258-261 (1993).
Strauss et al., “Germ line transmission of a yeast artificial chromosome spanning the murine a, (I) collagen locus”, Science, Vol. 259, pp.1904-1907 (1993).
Gilboa, E, Eglitis, M A, Kantoff, P W, Anderson, W F: Transfer and expression of cloned genes using retroviral vectors. BioTechniques 4(6):504-512, 1986.
Cregg J M, Vedvick T S, Raschke W C: Recent Advances in the Expression of Foreign Genes in Pichia pastoris, Bio/Technology 11:905-910,1993
Culver, 1998. Site-Directed recombination for repair of mutations in the human ADA gene. (Abstract) Antisense DNA & RNA based therapeutics, February, 1998, Coronado, Calif.
Huston et al, 1991 “Protein engineering of single-chain Fv analogs and fusion proteins” in Methods in Enzymology (J J Langone, ed.; Academic Press, New York, N.Y.) 203:46-88.
Johnson and Bird, 1991 “Construction of single-chain Fvb derivatives of monoclonal antibodies and their production in Escherichia coli in Methods in Enzymology (J J Langone, ed.; Academic Press, New York, N.Y.) 203:88-99.
Mernaugh and Mernaugh, 1995 “An overview of phage-displayed recombinant antibodies” in Molecular Methods In Plant Pathology (R P Singh and U S Singh, eds.; CRC Press Inc., Boca Raton, Fla.) pp. 359-365.

1. A method of promoting neurogenesis comprising the step of:
administering a therapeutic amount of a phosphodiesterase inhibitor compound to a patient in need of neurogenesis promotion.

2. The method according to claim 1, further including administering cellular therapy to the patient.

3. A compound for promoting neurogenesis comprising an effective amount of a phosphodiesterase inhibitor sufficient to promote neurogenesis.

4. The compound according to claim 3, further including a cellular therapy.

5. A neurogenesis promoter comprising a phosphodiesterase inhibitor in a pharmaceutically acceptable carrier.

6. The neurogenesis promoter according to claim 5, wherein said phosphodiesterase inhibitor augments nitric oxide in a tissue.

7. The neurogenesis promoter according to claim 6, wherein said phosphodiesterase inhibitor is sildenafil.

8. A method of augmenting the production of neurons by administering an effective amount of a phosphodiesterase inhibitor to a site in need of augmentation.

9. The method according to claim 8, further including administering cellular therapy to the site.

10. A method of increasing neurological function by administering an effective amount of a phosphodiesterase inhibitor to a patient.

11. The method according to claim 10, further including administering cellular therapy to the patient.

12. A method of increasing cognitive and neurological function by administering an effective amount of a phosphodiesterase inhibitor compound to a patient.

13. The method according to claim 12, further including administering cellular therapy to the patient.
